(19) United States
(12) Patent Application Publication
(10) Pub. No.: US 2004/0203002 A1
(43) Pub. Date: Oct. 14, 2004

(54) Determination of protein-DNA specificity

(76) Inventor: Yen Choo, London (GB)

Correspondence Address: ROBINS & PASTERNAK, 1731 EMBARCADERO ROAD, SUITE 230, PALO ALTO, CA, 94303, US

(21) Appl. No.: 10/370,984

(22) Filed: Feb. 21, 2003

Related U.S. Application Data
(63) Continuation of application No. PCT/US01/26435, filed on Aug. 24, 2001.
(60) Provisional application No. 60/227,900, filed on Aug. 25, 2000.

(51) Int. Cl. C12Q 1/68; G01N 33/53

(52) U.S. Cl. 435/6; 435/7.1

(57) ABSTRACT

A compound is contacted to an array that includes a plurality of capture probes. Probes to which the compound interact are identified to provide an interaction site profile. An exemplary compound is a polypeptide such as a transcription factor. An exemplary capture probe is a nucleic acid such as a double stranded DNA.

### BACKGROUND OF THE INVENTION

Over the next few years the sequences of hundreds of genomes will be completed. An important challenge in this post-genomic era of biology will be to dissect the regulatory networks that control gene expression. A key step in the regulation of these networks is the sequence-specific binding of transcription factors to their DNA recognition sites. The majority of documented transcription factor binding sites are located in non-protein-coding regions. Non-protein-coding regions make up about 97% of the human genome and are key in quantitative traits such as drug responses in the field of pharmacogenomics. The interpretation of such regions on a large scale will require systematic quantitative tools aimed specifically at DNA-protein interactions for thousands of proteins, DNA sites, and their allelic variants. A more complete understanding of these DNA-protein interactions will allow regulons to be interconnected by the identification of potential DNA binding sites for these trans-acting factors. This type of genomic data, taken together with other genomic data such as the analysis of mRNA expression patterns and the identification of putative protein-protein interactions, will permit a more comprehensive and quantitative mapping of the regulatory pathways within cells, as well as a deeper understanding of the potential functions of individual genes regulated by newly identified DNA binding sites (M. Schena, D. Shalon, R. W. Davis, P. O. Brown, Science 270, 467 (1995); P. Uetz, et al., Nature 403, 623-7 (2000)).

An understanding of the sequence specificity of DNA-protein interactions has resulted from studies of the effects of mutations in the DNA binding sites and the amino acid residues implicated in sequence-specific binding. The zinc finger transcription factors are one of the best understood families in terms of sequence-specific DNA binding. Rational zinc finger design using structure-based and database-guided approaches has permitted some progress in revealing certain rules that govern these discriminating contacts (J. R. Desjarlais, J. M. Berg, Proteins 12, 101 (1992); J. R. Desjarlais, J. M. Berg, Proc. Natl. Acad. Sci. U.S.A. 89, 7345 (1992); M. Elrod-Erickson, C. Pabo, J. Biol. Chem. 274, 19281 (1999); G. Jacobs, EMBO J. 11, 4507 (1992)). In addition, phage display has emerged as a powerful tool to select for zinc fingers that recognize given target DNA sites (E. J. Rebar, C. O. Pabo, Science 263, 671 (1994); H. A. Greisman, C. O. Pabo, Science 275, 657 (1997); A. Jamieson, S. Kim, J. Wells, Biochemistry 33, 5689 (1994); Y. Choo, A. Klug, Proc. Natl. Acad. Sci. U.S.A. 91, 11163 (1994); Y. Choo, I. Sanchez-Garcia, A. Klug, Nature 372, 642 (1994)). Although this technology has permitted millions of protein variants to be sampled simultaneously, the effects of individual mutants have had to be measured one at a time using nitrocellulose binding assays, gel mobility shift analysis, ELISA, Southwestern blotting, or reporter constructs (C. P. Woodbury, P. H. v. Hippel, Biochemistry 22, 4730 (1983); B. Bowen, J. Steinberg, U. K. Laemmli, H. Weintraub, Nucleic Acids Res. 8, 1 (1980); S. D. Hanes, R. Brent, Science 251,426 (1991); M. M. Garner, A. Revzin, Nucleic Acids Res. 9, 3047 (1981); Y. Choo, A. Klug, Nucleic Acids Res. 21,3341 (1993); A. Griffiths, et al., EMBO J. 13, 3245 (1994)). Because these methods are generally too laborious to be used for the analysis of a large number of DNA-protein interactions, it has not been possible to gather data on vast collections of variant DNA-protein pairings. While SELEX and ‘binding site signatures’ have permitted the sampling of multiple DNA binding sites for a given DNA binding protein, these in vitro selections provide only a partial view of binding site specificity, since only the tightest binding interactions are selected, while information on sub-optimal interactions is lost in the experimental process (C. Tuerk, L. Gold, Science 249, 505 (1990); Y. Choo, A. Klug, Proc. Natl. Acad. Sci. U.S.A. 91, 11168 (1994); J. Desjarlais, J. Berg, Proc. Natl. Acad. Sci. U.S.A. 91, 11099 (1994)).

### SUMMARY OF THE INVENTION

The current invention is based in part on the discovery that optimal and sub-optimal binding affinities for binding interactions can be detected using microarrays.

In one aspect, the current invention features a method of providing an interaction site profile. The method includes providing an array of capture probes, contacting the compound with the array, and identifying probes to which the compound interacts, thus providing an interaction site profile. The array includes a plurality of capture probes. Each capture probe is positionally distinguishable from the other probes, and includes a unique region.

In one embodiment, the interaction of the compound with the probe results in a covalent modification of the probe, e.g., a covalent bond of the probe can be broken or formed. In a preferred embodiment, the interaction of the compound with the capture probe is a binding interaction wherein neither the compound nor the probe has a covalent bond broken or formed.

In a preferred embodiment, the interaction site profile is a list of objects, each object representing a unique capture probe, and having an associated value, preferably a numerical value. The list can contain two, three, four, five, six, seven, eight, nine, ten, 15, 20, 50, 100, 1000 or more objects. In a preferred embodiment, each unique capture probe is represented by an object. In this embodiment, the list includes as many objects as unique capture probes. In another embodiment, the list includes the capture probes which interact with the compound. Thus, the list can contain only those capture probes for which an interaction was detected, or only those capture probes for which an interaction met a predetermined condition. Such a list has fewer objects as members than the number of unique capture probes. In a preferred embodiment, the interaction site profile is stored in computer memory, such as random access memory or flash memory, or on computer readable media, such as magnetic (e.g., a diskette, removable hard drive, or internal hard drive) or optical media (e.g., a compact disk (CD), DVD, or holographic media). A profile stored in this manner can be on a personal computer, server, e.g., a network server, or mainframe, and can be accessed from another device across a network, e.g., an intranet or internet. In another embodiment, the interaction site profile is printed on to a media such as a plastic, a paper or a label, e.g., as a bar code or variation thereof.

The value associated with each object of an interaction site profile can be obtained from a quantitative observation, or a qualitative observation, preferably a quantitative observation. In one embodiment, the associated value is a function of the amount of interaction between the compound and the probe. For example, the amount of interaction can be the amount of binding, the amount of probe modification, or affinity. In a preferred embodiment, the associated value is a function of the amount of binding between the compound and the probe. The value can be a function of the amount of a quantitative observation such as a fluorescent signal, a radioactive signal, or a phosphorescent signal of a contacted capture probe. The value can be provided by an instrument, e.g., a CCD camera. In one embodiment, the value is a function of the surface plasmon resonance at the site of a contacted capture probe. In a preferred embodiment, the associated values are adjusted for a background signal. In another embodiment, the associated value is a function of moles of bound compound. In yet another embodiment, the associated value is an affinity, relative affinity, apparent affinity, association constant, dissociation constant, logarithm of an affinity, or free energy for binding, of the compound for the capture probe. In a preferred embodiment, the associated values in the list are differ. In other words, the list contains more than one object, and i.e., the associated values of the objects in the list are not all the same. The values provide a range. The values can be distributed in the range. In some embodiments, the values can approximate a Poisson distribution. The list can contain objects whose associated values are zero, or null. The list can contain objects whose associated values are positive or negative. In one embodiment, the list does not contain any objects whose associated values are zero or null.

In a preferred embodiment, interaction site profiles are provided for a compound at varying concentrations of the compound, i.e. an interaction site profile is provided for a compound at a first concentration, at a second concentration, etc. In another preferred embodiment, interaction site profiles are provided for a compound for interaction with varying concentration of capture probes. For example, an array can have more than one unit, the compositions of the units being identical, but the first unit having the probes at a first concentration, and the second unit having the probes at a second concentration, etc. In yet another preferred embodiment, interaction site profiles are provide for a compound for various intervals after contacting the compound to the array. For example, a first profile can be provided after a first interval of time has elapsed after application, and a second profile can be provided after a second interval, etc.

The capture probes contain a unique region. In one embodiment, the unique region is an interaction site, an interaction site variant, or putative interaction site. In another embodiment, the unique region is random.

The array of the present invention can contain at least two, four, preferably 16, 64, 96, 128, 384, 1536, or more unique capture probes. In one embodiment, the array is a solid silica support. The plurality of nucleic acid probes can be stably attached to the support by a covalent bond. For example, a probe can be stably attached with a silane compound reactive with primary amines, or acrylamide moieties in the oligonucleotide or PCR productions, or an amino silane or polylysine or other polymer capable of UV crosslinking to single-stranded or double-stranded DNA. In a preferred embodiment, the array is a glass slide. The slide can be treated with an-activating agent, e.g., 1,4-diphenylene-diisothiocyanate (PDC).

In one embodiment, the capture probes are small organic chemicals, e.g., compounds with a molecular weight of 10,000, 5,000, 3,000, or 1,000 Daltons or less. The chemicals can be produced by combinatorial synthesis.

In a preferred embodiment, the capture probes on the array can be biological polymers such as nucleic acids, polypeptides, complex sugars, and combinations thereof. For example, a polypeptide can be covalently linked to a DNA or RNA. In one embodiment, the capture probes are polypeptides. The polypeptides can contain 2, 10, 20, 30, 50, or 100 or more amino acids. In a preferred embodiment, the polypeptides are antibodies. In a preferred embodiment, the capture probes are nucleic acids. The capture probes can be a deoxyribonucleic acid (DNA), a ribonucleic acid (RNA), a peptide nucleic acid (PNA), or any combination thereof. For example the capture probes can include a DNA, part of which is hybridized to part of an RNA. The capture probes can include a DNA ligated to an RNA or a nucleic acid chemically synthesized to include ribonucleotides and deoxyribonucleotides. In one embodiment, the capture probes are RNA. In a preferred embodiment, the capture probes are DNA, e.g., single-stranded DNA and double-stranded DNA. In a much preferred embodiment, the capture probes are double-stranded DNA, or consist substantially of double-stranded DNA. The capture probes can be at least about six, ten, 15, 20, 25, 30, 35, 40, 45, 50, 60, 70, 80, 90 or 100 or more basepairs in length. Preferably, the capture probes are about 15 to 90, or about 25 to 50 basepairs in length. In a preferred embodiment, the capture probes are about 15 to 30 basepairs in length. The unique region can comprise two, three, four, five, six, seven, eight, nine, ten, 15, 20, or more basepairs. The unique region can be contiguous, or can be interrupted by one or more common regions. In one embodiment, the DNA capture probes contain fragments of genomic DNA, preferably non-coding regions of genomic DNA, more preferably regulatory regions of genomic DNA, most preferably promoters and enhancers.

In embodiments in which the monitored interaction between the compound and the capture probes is a binding interaction, the unique region is preferably a binding site. In one such embodiment, the array includes all possible combinations of natural basepair substitutions (e.g., adenine-thymidine, thymidine-adenine, guanine-cytosine, cytosine-guanine basepairs) at greater than two basepairs of the interaction site. Thus, the array includes at least 64 unique capture probes. In another such embodiment, the array includes all possible combinations of natural basepair substitutions at greater than three basepairs of the interaction site, i.e. 256 unique capture probes.

The compound for which the interaction site profile is generated can be a polypeptide, a peptoid, a PNA, or a chemically modified polypeptide. In a much preferred embodiment the compound is a polypeptide. The polypeptide can be a nucleic acid binding protein. In one embodiment, the polypeptide is an RNA contacting protein such as a splicing factor, a ribosomal protein, a viral protein, an RNA modification enzyme, a translation factor, and the like. In a preferred embodiment, the polypeptide is a DNA contacting protein such as a transcription factor, a replication factor, a telomere binding protein, a centromere binding protein, a restriction modification enzyme, a DNA methylase, DNA repair protein, a single-stranded DNA binding protein, a recombination protein and the like. In a much preferred embodiment, the polypeptide is a transcription factor. The transcription factor can bind a double stranded DNA sequence with an affinity of 10 mM, 1 mM, 100 nM or less, preferably 10 nM or less, 1 nM or less, and even more preferably 100 pM or less. The transcription factor can be selected from the group consisting of homeodomains, helix-turn-helix motif proteins, beta-sheets, leucine zippers, steroid receptors, zinc fingers and histones. In one embodiment, the polypeptide is a zinc finger polypeptide. In another embodiment, the polypeptide is modified or combined with natural and exotic chemical ligands. In yet another embodiment, the compound for which the interaction site profile is generated includes more than one polypeptide, i.e., polypeptides are combined.

In a preferred embodiment, the polypeptide is covalently attached to bacteriophage, e.g., a T7 phage, a lambdoid phage, or a filamentous phage. Preferably, the polypeptide is covalent attached to a filamentous phage such as fd or M13. The polypeptide can be covalently fused to a coat protein by constructing a fusion gene with the gene encoding the polypeptide an the viral coat protein gene, e.g., filamentous phage gene VIII or gene III. In another preferred embodiment, the polypeptide is covalently attached to green fluorescent protein (GFP), or a variant thereof (such as enhanced GFP, CFP, BFP, and the like). The polypeptide can be covalently attached by constructing a fusion gene. In yet another embodiment, the polypeptide is linked with an unrelated sequence, e.g., a fusion protein, purification handle, or epitope tag. Useful examples of such unrelated sequences include maltose binding protein, glutathione-S-transferase, chitin binding protein, thioredoxin, hexa-histidine (or 6-His), the “FLAG tag”, the myc epitope, and the hemagglutinin epitope.

In one embodiment, the polypeptide contains a detectable label. The detectable label can be a radiolabel. Preferably, the detectably label is a fluorescent label, e.g., malachite green, Oregon green, Texas Red, Congo Red, Cy3, SybrGreen I, or R-phycoerythrin. In another embodiment, the polypeptide is contacted with an antibody. The antibody can contact the protein directly or can contact a covalently attached tag, e.g., a moiety mentioned above.

In a preferred embodiment, the polypeptide is a variant of a natural counterpart. The variant can have at least one amino acid difference from the natural counterpart. Preferably the differing amino acid is located within 50 Ångstroms, 20 Ångstroms, or 10 Ångstroms or less of the bound nucleic acid in a structural model, e.g., a model built from X-ray diffraction data, NMR restraint data, or another homology model.

In another aspect, the present invention features a method of evaluating a plurality of compounds. The method includes providing the compounds, providing an array of capture probes, contacting each compound with an array, identifying capture probes which interact with each compound to generate a interaction site profile for each compound, then comparing the interaction site profiles to evaluate the compounds.

In one embodiment, the interaction of the compound with the probe results in a covalent modification of the probe, e.g., a covalent bond of the probe can be broken or formed. In a preferred embodiment, the interaction of the compound with the capture probe is a binding interaction wherein neither the compound nor the probe has a covalent bond broken or formed.

In a preferred embodiment, the interaction site profile is a list of objects, each object representing a unique capture probe, and having an associated value, preferably a numerical value. The list can contain two, three, four, five, six, seven, eight, nine, ten, 15, 20, 50, 100, 1000 or more objects. In a preferred embodiment, each unique capture probe is represented by an object. In this embodiment, the list includes as many objects as unique capture probes. In another embodiment, the list includes the capture probes which interact with the compound. Thus, the list can contain only those capture probes for which an interaction was detected, or only those capture probes for which an interaction met a predetermined condition. Such a list has fewer objects as members than the number of unique capture probes. In a preferred embodiment, the interaction site profile is stored in computer memory, such as random access memory or flash memory, or on computer readable media, such as magnetic (e.g., a diskette, removable hard drive, or internal hard drive) or optical media (e.g., a compact disk (CD), DVD, or holographic media). A profile stored in this manner can be on a personal computer, server, e.g., a network server, or mainframe, and can be accessed from another device across a network, e.g., an intranet or internet. In another embodiment, the interaction site profile is printed on to a media such as a plastic, a paper or a label, e.g., as a bar code or variation thereof.

The value associated with each object of an interaction site profile can be obtained from a quantitative observation, or a qualitative observation, preferably a quantitative observation. In one embodiment, the associated value is a function of the amount of interaction between the compound and the probe. For example, the amount of interaction can be the amount of binding, the amount of probe modification, or affinity. In a preferred embodiment, the associated value is a function of the amount of binding between the compound and the probe. The value can be a function of the amount of a quantitative observation such as a fluorescent signal, a radioactive signal, or a phosphorescent signal of a contacted capture probe. The value can be provided by an instrument, e.g., a CCD camera. In one embodiment, the value is a function of the surface plasmon resonance at the site of a contacted capture probe. In a preferred embodiment, the associated values are adjusted for a background signal. In another embodiment, the associated value is a function of moles of bound compound. In yet another embodiment, the associated value is an affinity, relative affinity, apparent affinity, association constant, dissociation constant, logarithm of an affinity, or free energy for binding, of the compound for the capture probe. In a preferred embodiment, the associated values in the list are differ. In other words, the list contains more than one object, and i.e., the associated values of the objects in the list are not all the same. The values provide a range. The values can be distributed in the range. In some embodiments, the values can approximate a Poisson distribution. The list can contain objects whose associated values are zero, or null. The list can contain objects whose associated values are positive or negative. In one embodiment, the list does not contain any objects whose associated values are zero or null.

In a preferred embodiment, interaction site profiles are provided for a compound at varying concentrations of the compound, i.e. an interaction site profile is provided for a compound at a first concentration, at a second concentration, etc. In another preferred embodiment, interaction site profiles are provided for a compound for interaction with varying concentration of capture probes. For example, an array can have more than one unit, the compositions of the units being identical, but the first unit having the probes at a first concentration, and the second unit having the probes at a second concentration, etc. In yet another preferred embodiment, interaction site profiles are provide for a compound for various intervals after contacting the compound to the array. For example, a first profile can be provided after a first interval of time has elapsed after application, and a second profile can be provided after a second interval, etc.

The capture probes contain a unique region. In one embodiment, the unique region is an interaction site, an interaction site variant, or putative interaction site. In another embodiment, the unique region is random.

The array of the present invention can contain at least two, four, preferably 16, 64, 96, 128, 384, 1536, or more unique capture probes. In one embodiment, the array is a solid silica support. The plurality of nucleic acid probes can be stably attached to the support by a covalent bond. For example, a probe can be stably attached with a silane compound reactive with primary amines, or acrylamide moieties in the oligonucleotide or PCR productions, or an amino silane or polylysine or other polymer capable of UV crosslinking to single-stranded or double-stranded DNA. In a preferred embodiment, the array is a glass slide. The slide can be treated with an activating agent, e.g., 1,4-diphenylene-diisothiocyanate (PDC).

In one embodiment, the capture probes are small organic chemicals, e.g., compounds with a molecular weight of 10,000, 5,000, 3,000, or 1,000 Daltons or less. The chemicals can be produced by combinatorial synthesis.

In a preferred embodiment, the capture probes on the array can be biological polymers such as nucleic acids, polypeptides, complex sugars, and combinations thereof. For example, a polypeptide can be covalently linked to a DNA or RNA. In one embodiment, the capture probes are polypeptides. The polypeptides can contain 2, 10, 20, 30, 50, or 100 or more amino acids. In a preferred embodiment, the polypeptides are antibodies. In a preferred embodiment, the capture probes are nucleic acids. The capture probes can be a deoxyribonucleic acid (DNA), a ribonucleic acid (RNA), a peptide nucleic acid LPNA), or any combination thereof. For example the capture probes can include a DNA, part of which is hybridized to part of an RNA. The capture probes can include a DNA ligated to an RNA or a nucleic acid chemically synthesized to include ribonucleotides and deoxyribonucleotides. In one embodiment, the capture probes are RNA. In a preferred embodiment, the capture probes are DNA, e.g., single-stranded DNA and double-stranded DNA.

In a much preferred embodiment, the capture probes are double-stranded DNA, or consist substantially of double-stranded DNA. The capture probes can be at least about six, ten, 15, 20, 25, 30, 35, 40,45, 50,60, 70, 80, 90 or 100 or more basepairs in length. Preferably, the capture probes are about 15 to 90, or about 25 to 50 basepairs in length. In a preferred embodiment, the capture probes are about 15 to 30 basepairs in length. The unique region can comprise two, three, four, five, six, seven, eight, nine, ten, 15, 20, or more basepairs. The unique region can be contiguous, or can be interrupted by one or more common regions. In one embodiment, the DNA capture probes contain fragments of genomic DNA, preferably non-coding regions of genomic DNA, more preferably regulatory regions of genomic DNA, most preferably promoters and enhancers.

In embodiments in which the monitored interaction between the compound and the capture probes is a binding interaction, the unique region is preferably a binding site. In one such embodiment, the array includes all possible combinations of natural basepair substitutions (e.g., adenine-thymidine, thymidine-adenine, guanine-cytosine, cytosine-guanine basepairs) at greater than two basepairs of the interaction site. Thus, the array includes at least 64 unique capture probes. In another such embodiment, the array includes all possible combinations of natural basepair substitutions at greater than three basepairs of the interaction site, i.e. 256 unique capture probes.

The compound for which the interaction site profile is generated can be a polypeptide, a peptoid, a PNA, or a chemically modified polypeptide. In a much preferred embodiment the compound is a polypeptide. The polypeptide can be a nucleic acid binding protein. In one embodiment, the polypeptide is an RNA contacting protein such as a splicing factor, a ribosomal protein, a viral protein, an RNA modification enzyme, a translation factor, and the like. In a preferred embodiment, the polypeptide is a DNA contacting protein such as a transcription factor, a replication factor, a telomere binding protein, a centromere binding protein, a restriction modification enzyme, a DNA methylase, DNA repair protein, a single-stranded DNA binding protein, a recombination protein and the like. In a much preferred embodiment, the polypeptide is a transcription factor. The transcription factor can bind a double stranded DNA sequence with an affinity of 10 mM, 1 mM, 100 nM or less, preferably 10 nM or less, 1 nM or less, and even more preferably 100 pM or less. The transcription factor can be selected from the group consisting of homeodomains, helix-tun-helix motif proteins, beta-sheets, leucine zippers, steroid receptors, zinc fingers and histones. In one embodiment, the polypeptide is a zinc finger polypeptide. In another embodiment, the polypeptide is modified or combined with natural and exotic chemical ligands. In yet another embodiment, the compound for which the interaction site profile is generated includes more than one polypeptide, i.e., polypeptides are combined.

In a preferred embodiment, the polypeptide is covalently attached to bacteriophage, e.g., a T7 phage, a lambdoid phage, or a filamentous phage. Preferably, the polypeptide is covalent attached to a filamentous phage such as fd or M13. The polypeptide can be covalently fused to a coat protein by constructing a fusion gene with the gene encoding the polypeptide an the viral coat protein gene, e.g., filamentous phage gene VIII or gene III. In another preferred embodiment, the polypeptide is covalently attached to green fluorescent protein (GFP), or a variant thereof (such as enhanced GFP, CFP, BFP, and the like). The polypeptide can be covalently attached by constructing a fusion gene. In yet another embodiment, the polypeptide is linked with an unrelated sequence, e.g., a fusion protein, purification handle, or epitope tag. Useful examples of such unrelated sequences include maltose binding protein, glutathione-S-transferase, chitin binding protein, thioredoxin, hexa-histidine (or 6-His), the “FLAG tag”, the myc epitope, and the hemagglutinin epitope.

In one embodiment, the polypeptide contains a detectable label. The detectable label can be a radiolabel. Preferably, the detectably label is a fluorescent label, e.g., malachite green, Oregon green, Texas Red, Congo Red, Cy3, SybrGreen I, or R-phycoerythrin. In another embodiment, the polypeptide is contacted with an antibody. The antibody can contact the protein directly or can contact a covalently attached tag, e.g., a moiety mentioned above.

In a preferred embodiment, the polypeptide is a variant of a natural counterpart. The variant can have at least one amino acid difference from the natural counterpart. Preferably the differing amino acid is located within 50 Ångstroms, 20 Ångstroms, or 10 Ångstroms or less of the bound nucleic acid in a structural model, e.g., a model built from X-ray diffraction data, NMR restraint data, or another homology model.

In one embodiment, the compounds being compared are contacted to a single array. Preferably, the compound are differentially labeled in order to measure the amount of interaction of each with the probes. Alternatively, a first compound is contacted to the array, interactions are observed, then the second compound is contacted, and interactions are observed. A comparison can be made between the second set of observations and the first set, e.g., the first dataset can be subtracted from the second. In a preferred embodiment, the compounds are compared by contacting each compound to its own array to obtain profiles for each. Preferably, the arrays are of the same design and composition. Even more preferably, the arrays are produced from the same process, source material, and batch.

In a preferred embodiment, the method also include comparing two interaction site profiles using a difference profile. A difference profile includes a list of objects which are common to both interaction site profiles, and values associated with each object. The value is a function of the values associated with the corresponding objects in the two interaction site profiles being compared. Preferably, two objects correspond if they reference capture probes with the same composition and structure, e.g., the same nucleic acid sequence. In a preferred embodiment, the associated value for an object, is calculated as the arithmetic difference of the value associated with the object in the first profile from the value associated with the object in the second profile. In one embodiment, the difference profile is reduced to a single score, e.g., the sum of the squares of its associated values. In another embodiment, two interaction site profiles are compared by providing a value which is a function of at least one of the values in the difference profile. For example, the value can be an average of the values for two objects which represent probes with a desired target structure, e.g., a desired sequence.

In another preferred embodiment, two interaction site profiles are compared by comparing one or a plurality of the associated values in the two interaction site profiles.

For example, the relevant associated values can refer to one probe for which an interaction is desirable, and to another probe for which an interaction is undesirable. In another example, the relevant associated values can refer to one probe for which an interaction is desirable, and to another probe for which an interaction is of intermediate magnitude is acceptable. Such comparisons as described herein, and other variations which will be apparent to a skilled thereof allow for the comparison of interactions based on suboptimal sites.

In embodiments, wherein interaction site profiles only include those capture probes for which an interaction was detected, or only those capture probes for which an interaction met a predetermined condition, two profiles can be compared by comparing the objects in the profile. For example, the profiles can be rank ordered. Two profiles can be considered similar if the same object has the first rank in both profiles. In another example, the profiles is ranked, and the first two, three, four, five, or six or more objects are selected. The profiles can be considered similar, if the same objects or the same subset of objects are selected. Such a method and variations thereof provide for comparison of interactions with suboptimal sites. In yet another example, rank ordered profiles can be considered similar if they have the same object in the first rank position and the same object in the last rank position. Other variations will be apparent to a skilled artisan.

In another embodiment, two interaction site profiles are compared to each other indirectly, by comparison to a reference profile, for example, a predetermined profile or a profile of a characterized compound, a profile of a characterized collection (e.g., library) or an arbitrary profile. Any means of comparison described herein can be used to compare the first interaction site profile with the reference profile. Preferably, the second interaction site profile is compared by the same means. The first and second profile can be considered similar if they both meet or both fail a preselected criterion with regard to the comparisons to the reference profile.

In another embodiment, a first interaction site profile is compared to more than one interaction site profile, e.g., profiles for a library of compounds, a database of profiles, a set of target profiles, and the like. Any means of comparison described herein can be used to compare the first interaction site profile with the plurality of profiles. In a preferred embodiment, the first interaction site profile is compared sequentially or concurrently with each profile of the plurality. A statistical operation, e.g., averaging, variance, etc., is used to synthesize the results of the individual comparisons to arrive at a criterion of comparing the first profile with the plurality of profiles. In another preferred embodiment, the plurality of profiles is merged into a single profile, for example using a statistical operation, and the first profile is compared to the merged profile using methods described herein.

In a much preferred embodiment, the comparison is executed by a computer system programmed by a computer readable code. The computer system can include a database of profiles for comparison. The computer system can further include a graphical user interface which allows a user to select profiles for comparison and a method for comparison.

In another aspect, the invention features a method of evaluating a first polypeptide. The method includes providing the first polypeptide for evaluation and one or more reference polypeptides. The method further includes obtaining interaction site profiles for the first polypeptide and for one or more reference polypeptides. The interaction site profiles are provided by: providing an array of a plurality of nucleic acid probes, wherein each of the probes in the plurality is positionally distinguishable from other probes of the plurality, and wherein each positionally distinguishable probe includes a unique region; contacting the first polypeptide, and one or more reference polypeptides with the array of probes; and identifying probes to which the polypeptide interacts to thereby provide interaction site profiles for the first polypeptide, and one or more reference polypeptides. The method can still further include comparing the interaction site profile of the first polypeptide with the interaction site profile of each reference polypeptide to thereby evaluate the first polypeptide.

In one embodiment, the interaction of the compound with the probe results in a covalent modification of the probe, e.g., a covalent bond of the probe can be broken or formed. In a preferred embodiment, the interaction of the compound with the capture probe is a binding interaction wherein neither the compound nor the probe has a covalent bond broken or formed.

In a preferred embodiment, the interaction site profile is a list of objects, each object representing a unique capture probe, and having an associated value, preferably a numerical value. The list can contain two, three, four, five, six, seven, eight, nine, ten, 15,20, 50, 100, 1000 or more objects. In a preferred embodiment, each unique capture probe is represented by an object. In this embodiment, the list includes as many objects as unique capture probes. In another embodiment, the list includes the capture probes which interact with the compound. Thus, the list can contain only those capture probes for which an interaction was detected, or only those capture probes for which an interaction met a predetermined condition. Such a list has fewer objects as members than the number of unique capture probes. In a preferred embodiment, the interaction site profile is stored in computer memory, such as random access memory or flash memory, or on computer readable media, such as magnetic (e.g., a diskette, removable hard drive, or internal hard drive) or optical media (e.g., a compact disk (CD), DVD, or holographic media). A profile stored in this manner can be on a personal computer, server, e.g., a network server, or mainframe, and can be accessed from another device across a network, e.g., an intranet or internet. In another embodiment, the interaction site profile is printed on to a media such as a plastic, a paper or a label, e.g., as a bar code or variation thereof.

The value associated with each object of an interaction site profile can be obtained from a quantitative observation, or a qualitative observation, preferably a quantitative observation. In one embodiment, the associated value is a function of the amount of interaction between the compound and the probe. For example, the amount of interaction can be the amount of binding, the amount of probe modification, or affinity. In a preferred embodiment, the associated value is a function of the amount of binding between the compound and the probe. The value can be a function of the amount of a quantitative observation such as a fluorescent signal, a radioactive signal, or a phosphorescent signal of a contacted capture probe. The value can be provided by an instrument, e.g., a CCD camera. In one embodiment, the value is a function of the surface plasmon resonance at the site of a contacted capture probe. In a preferred embodiment, the associated values are adjusted for a background signal. In another embodiment, the associated value is a function of moles of bound compound. In yet another embodiment, the associated value is an affinity, relative affinity, apparent affinity, association constant, dissociation constant, logarithm of an affinity, or free energy for binding, of the compound for the capture probe. In a preferred embodiment, the associated values in the list are differ. In other words, the list contains more than one object, and i.e., the associated values of the objects in the list are not all the same. The values provide a range. The values can be distributed in the range. In some embodiments, the values can approximate a Poisson distribution. The list can contain objects whose associated values are zero, or null. The list can contain objects whose associated values are positive or negative. In one embodiment, the list does not contain any objects whose associated values are zero or null.

In a preferred embodiment, interaction site profiles are provided for a compound at varying concentrations of the compound, i.e. an interaction site profile is provided for a compound at a first concentration, at a second concentration, etc. In another preferred embodiment, interaction site profiles are provided for a compound for interaction with varying concentration of capture probes. For example, an array can have more than one unit, the compositions of the units being identical, but the first unit having the probes at a first concentration, and the second unit having the probes at a second concentration, etc. In yet another preferred embodiment, interaction site profiles are provide for a compound for various intervals after contacting the compound to the array. For example, a first profile can be provided after a first interval of time has elapsed after application, and a second profile can be provided after a second interval, etc.

The capture probes contain a unique region. In one embodiment, the unique region is an interaction site, an interaction site variant, or putative interaction site. In another embodiment, the unique region is random.

The array of the present invention can contain at least two, four, preferably 16, 64, 96, 128, 384, 1536, or more unique capture probes. In one embodiment, the array is a solid silica support. The plurality of nucleic acid probes can be stably attached to the support by a covalent bond. For example, a probe can be stably attached with a silane compound reactive with primary amines, or acrylamide moieties in the oligonucleotide or PCR productions, or an amino silane or polylysine or other polymer capable of UV crosslinking to single-stranded or double-stranded DNA. In a preferred embodiment, the array is a glass slide. The slide can be treated with an activating agent, e.g., 1,4-diphenylene-diisothiocyanate (PDC).

In one embodiment, the capture probes are small organic chemicals, e.g., compounds with a molecular weight of 10,000, 5,000, 3,000, or 1,000 Daltons or less. The chemicals can be produced by combinatorial synthesis.

The capture probes are polypeptides. The polypeptides can contain 2, 10, 20, 30, 50, or 100 or more amino acids. In a preferred embodiment, the polypeptides are antibodies. In a preferred embodiment, the capture probes are nucleic acids. The capture probes can be a deoxyribonucleic acid (DNA), a ribonucleic acid (RNA), a peptide nucleic acid (PNA), or any combination thereof. For example the capture probes can include a DNA, part of which is hybridized to part of an RNA. The capture probes can include a DNA ligated to an RNA or a nucleic acid chemically synthesized to include ribonucleotides and deoxyribonucleotides. In one embodiment, the capture probes are RNA. In a preferred embodiment, the capture probes are DNA, e.g., single-stranded DNA and double-stranded DNA.

In a much preferred embodiment, the capture probes are double-stranded DNA, or consist substantially of double-stranded DNA. The capture probes can be at least about six, ten, 15, 20, 25, 30, 35, 40, 45, 50, 60, 70, 80, 90 or 100 or more basepairs in length. Preferably, the capture probes are about 15 to 90, or about 25 to 50 basepairs in length. In a preferred embodiment, the capture probes are about 15 to 30 basepairs in length. The unique region can comprise two, three, four, five, six, seven, eight, nine, ten, 15, 20, or more basepairs. The unique region can be contiguous, or can be interrupted by one or more common regions. In one embodiment, the DNA capture probes contain fragments of genomic DNA, preferably non-coding regions of genomic DNA, more preferably regulatory regions of genomic DNA, most preferably promoters and enhancers.

In embodiments in which the monitored interaction between the compound and the capture probes is a binding interaction, the unique region is preferably a binding site. In one such embodiment, the array includes all possible combinations of natural basepair substitutions (e.g., adenine-thymidine, thymidine-adenine, guanine-cytosine, cytosine-guanine basepairs) at greater than two basepairs of the interaction site. Thus, the array includes at least 64 unique capture probes. In another such embodiment, the array includes all possible combinations of natural basepair substitutions at greater than tee basepairs of the interaction site, i.e. 256 unique capture probes.

The compound for which the interaction site profile is generated can be a polypeptide, a peptoid, a PNA, or a chemically modified polypeptide. In a much preferred embodiment the compound is a polypeptide. The polypeptide can be a nucleic acid binding protein. In one embodiment, the polypeptide is an RNA contacting protein such as a splicing factor, a ribosomal protein, a viral protein, an RNA modification enzyme, a translation factor, and the like. In a preferred embodiment, the polypeptide is a DNA contacting protein such as a transcription factor, a replication factor, a telomere binding protein, a centromere binding protein, a restriction modification enzyme, a DNA methylase, DNA repair protein, a single-stranded DNA binding protein, a recombination protein and the like. In a much preferred embodiment, the polypeptide is a transcription factor. The transcription factor can bind a double stranded DNA sequence with an affinity of 10 mM, 1 mM, 100 nM or less, preferably 10 nM or less, 1 nM or less, and even more preferably 100 pM or less. The transcription factor can be selected from the group consisting of homeodomains, helix-turn-helix motif proteins, beta-sheets, leucine zippers, steroid receptors, zinc fingers and histones. In one embodiment, the polypeptide is a zinc finger polypeptide. In another embodiment, the polypeptide is modified or combined with natural and exotic chemical ligands. In yet another embodiment, the compound for which the interaction site profile is generated includes more than one polypeptide, i.e., polypeptides are combined.

In a preferred embodiment, the polypeptide is covalently attached to bacteriophage, e.g., a T7 phage, a lambdoid phage, or a filamentous phage. Preferably, the polypeptide is covalent attached to a filamentous phage such as fd or M13. The polypeptide can be covalently fused to a coat protein by constructing a fusion gene with the gene encoding the polypeptide an the viral coat protein gene, e.g., filamentous phage gene VIII or gene III. In another preferred embodiment, the polypeptide is covalently attached to green fluorescent protein (GFP), or a variant thereof (such as enhanced GFP, CFP, BFP, and the like). The polypeptide can be covalently attached by constructing a fusion gene. In yet another embodiment, the polypeptide is linked with an unrelated sequence, e.g., a fusion protein, purification handle, or epitope tag. Useful examples of such unrelated sequences include maltose binding protein, glutathione-S-transferase, chitin binding protein, thioredoxin, hexa-histidine (or 6-His), the “FLAG tag”, the myc epitope, and the hemagglutinin epitope.

In one embodiment, the polypeptide contains a detectable label. The detectable label can be a radiolabel. Preferably, the detectably label is a fluorescent label, e.g., malachite green, Oregon green, Texas Red, Congo Red, Cy3, SybrGreen I, or R-phycoerythrin. In another embodiment, the polypeptide is contacted with an antibody. The antibody can contact the protein directly or can contact a covalently attached tag, e.g., a moiety mentioned above.

In a preferred embodiment, the polypeptide is a variant of a natural counterpart. The variant can have at least one amino acid difference from the natural counterpart. Preferably the differing amino acid is located within 50 Ångstroms, 20 Ångstroms, or 10 Ångstroms or less of the bound nucleic acid in a structural model, e.g., a model built from X-ray diffraction data, NMR restraint data, or another homology model.

In one embodiment, the compounds being compared are contacted to a single army. Preferably, the compound are differentially labeled in order to measure the amount of interaction of each with the probes. Alternatively, a first compound is contacted to the array, interactions are observed, then the second compound is contacted, and interactions are observed. A comparison can be made between the second set of observations and the first set, e.g., the first dataset can be subtracted from the second. In a preferred embodiment, the compounds are compared by contacting each compound to its own array to obtain profiles for each. Preferably, the arrays are of the same design and composition. Even more preferably, the arrays are produced from the same process, source material, and batch.

In a preferred embodiment, the method also include comparing two interaction site profiles using a difference profile. A difference profile includes a list of objects which are common to both interaction site profiles, and values associated with each object. The value is a function of the values associated with the corresponding objects in the two interaction site profiles being compared. Preferably, two objects correspond if they reference capture probes with the same composition and structure, e.g., the same nucleic acid sequence. In a preferred embodiment, the associated value for an object, is calculated as the arithmetic difference of the value associated with the object in the first profile from the value associated with the object in the second profile. In one embodiment, the difference profile is reduced to a single score, e.g., the sum of the squares of its associated values. In another embodiment, two interaction site profiles are compared by providing a value which is a function of at least one of the values in the difference profile. For example, the value can be an average of the values for two objects which represent probes with a desired target structure, e.g., a desired sequence.

In another preferred embodiment, two interaction site profiles are compared by comparing one or a plurality of the associated values in the two interaction site profiles.

For example, the relevant associated values can refer to one probe for which an interaction is desirable, and to another probe for which an interaction is undesirable. In another example, the relevant associated values can refer to one probe for which an interaction is desirable, and to another probe for which an interaction is of intermediate magnitude is acceptable. Such comparisons as described herein, and other variations which will be apparent to a skilled thereof allow for the comparison of interactions based on suboptimal sites.

In embodiments, wherein interaction site profiles only include those capture probes for which an interaction was detected, or only those capture probes for which an interaction met a predetermined condition, two profiles can be compared by comparing the objects in the profile. For example, the profiles can be rank ordered. Two profiles can be considered similar if the same object has the first rank in both profiles. In another example, the profiles is ranked, and the first two, three, four, five, or six or more objects are selected. The profiles can be considered similar, if the same objects or the same subset of objects are selected. Such a method and variations thereof provide for comparison of interactions with suboptimal sites. In yet another example, rank ordered profiles can be considered similar if they have the same object in the first rank position and the same object in the last rank position. Other variations will be apparent to a skilled artisan.

In another embodiment, two interaction site profiles are compared to each other indirectly, by comparison to a reference profile, for example, a predetermined profile or a profile of a characterized compound, a profile of a characterized collection (e.g., library) or an arbitrary profile. Any means of comparison described herein can be used to compare the first interaction site profile with the reference profile. Preferably, the second interaction site profile is compared by the same means. The first and second profile can be considered similar if they both meet or both fail a preselected criterion with regard to the comparisons to the reference profile.

In another embodiment, a first interaction site profile is compared to more than one interaction site profile, e.g., profiles for a library of compounds, a database of profiles, a set of target profiles, and the like. Any means of comparison described herein can be used to compare the first interaction site profile with the plurality of profiles. In a preferred embodiment, the first interaction site profile is compared sequentially or concurrently with each profile of the plurality. A statistical operation, e.g., averaging, variance, etc., is used to synthesize the results of the individual comparisons to arrive at a criterion of comparing the first profile with the plurality of profiles. In another preferred embodiment, the plurality of profiles is merged into a single profile, for example using a statistical operation, and the first profile is compared to the merged profile using methods described herein.

In a much preferred embodiment, the comparison is executed by a computer system programmed by a computer readable code. The computer system can include a database of profiles for comparison. The computer system can further include a graphical user interface which allows a user to select profiles for comparison and a method for comparison.

In a preferred embodiment, the method of evaluating a first polypeptide further includes identifying a selected reference polypeptide from the plurality of reference polypeptides such that the interaction site profiles of the selected reference polypeptides meets a predetermined level of similarity with the interaction site profile of the first polypeptide. The function of selected reference polypeptide is then assigned to the first polypeptide. The profile of the first polypeptide can be stored on a first computer system while the profiles of the one or more reference polypeptides can be stored on the same or on a second computer system, e.g., as a database. The method can further include communications across a computer network between the first and second computer system to thereby evaluate the first polypeptide.

In one embodiment, the reference polypeptides largely include polypeptides that are homologous, or of the same protein family, e.g., homeodomains, zinc fingers. In another embodiment, the reference polypeptides largely include polypeptides from the same species, e.g., maize, rice, Arabidopsis, E. coli, B. subtilis, Drosophil Saccharomyces cerevisiae, mouse, or human. In yet another embodiment, the reference polypeptides largely include polypeptides implicated in a disease or disease, e.g., a set polypeptides overexpressed in cancer cells, a set polypeptides related to human genetic disorders, a set of polypeptides implicated in hypertension, stroke, metastasis, obesity, and the like.

In another aspect, the present invention features a method of selecting a polypeptide. The method includes: providing a plurality of polypeptides; contacting the plurality with a substrate comprising target nucleic acid; isolating a selected population from the plurality; identifying an interaction site profile for the select population; evaluating the interaction site profile for a predetermined condition; and isolating a polypeptide from the selected population to thereby select a polypeptide. The interaction site profile is identified by: providing an array of a plurality of nucleic acid probes, wherein each of the probes in the plurality is positionally distinguishable from other probes of the plurality, and wherein each positionally distinguishable probe includes a unique region which corresponds to a binding site for the polypeptide, and wherein at least one probe of the plurality comprises the target sequence; contacting the selected population with the array; and identifying probes to which the selected population interacts thereby identifying the interaction site profile of the selected population.

In one embodiment, a selected population is isolated from the plurality by isolating those polypeptides of the plurality which interact with the substrate. For example, the polypeptides can be released from the substrate applying elution conditions, e.g., by altering ionic strength or pH. Selected populations can be provided by treating with intermediate elution conditions. In another embodiment, a selected population is isolated from the plurality by isolating those polypeptides of the plurality which fail to interact with the substrate. For example, the polypeptides can be obtained from the “flow-through” material after contact with the substrate.

In a preferred embodiment, if the interaction site profile for a selected population does not meet a predetermined condition, the cycle of contacting the substrate, isolating a (second) selected population, and evaluating its interaction site profile is repeated. The cycle can be repeated until the predetermined conditions is met. In another embodiment, the cycle is repeated until no change in the interaction site profile is observed. In yet another embodiment, the selected population can be further modified between cycles. For example, if the polypeptides in a selected population are attached the nucleic acid sequence which encodes them (e.g., a bacteriophage library, or an RNA display library or variations thereof), the nucleic acid can be mutagenized, e.g., by mutagenic PCR, or by sexual PCR to produce a modified selected population of polypeptides. Alternatively the polypeptides can be altered by expression from nucleic acids in systems using altered and/or expanded genetic codes.

In one embodiment, the predetermined condition is a binding affinity for a selected target compound, the compound being one of the capture probes. In another embodiment, the predetermined condition is a first predetermined affinity for a first target and a second predefined affinity for a second target, etc. In yet another embodiment, the predetermined condition is itself a interaction site profile, e.g., one designed for a specific application.

The plurality of polypeptides can be proteins from a provided sample, from a patient sample, or from a cell lysate. In a preferred embodiment, the plurality of polypeptides is from an expression library, e.g., a genomic or cDNA library, or a specialized library of nucleic acid binding proteins. In a much preferred embodiment, the plurality of polypeptides are variants of a progenitor polypeptide. The variants can be generated by a method including cassette mutagenesis, PCR mutagenesis, and the like. In a preferred embodiment, the variants have amino acid differences from the progenitor in at least one amino acid that is within 10 Ångstroms of the nucleic acid binding interface, e.g., an interface identified by a structural model or by mutagenesis. In a preferred embodiment, such variants are in a transcription factor.

In one embodiment, the target sequence is a degenerate nucleic acid and the substrate includes more than one species of nucleic acid.

In another aspect, the invention features a method of selecting a polypeptide which meets a predetermined criterion. The method includes: providing a plurality of polypeptides, identifying the interaction site profile of each polypeptide variant of the plurality by the methods described above; selecting the polypeptide whose interaction site profile meets the predetermined criterion to thereby select a polypeptide which meets a predetermined criterion.

In one embodiment, the interaction of the compound with the probe results in a covalent modification of the probe, e.g., a covalent bond of the probe can be broken or formed. In a preferred embodiment, the interaction of the compound with the capture probe is a binding interaction wherein neither the compound nor the probe has a covalent bond broken or formed.

In a preferred embodiment, the interaction site profile is a list of objects, each object representing a unique capture probe, and having an associated value, preferably a numerical value. The list can contain two, three, four, five, six, seven, eight, nine, ten, 15, 20, 50, 100, 1000 or more objects. In a preferred embodiment, each unique capture probe is represented by an object. In this embodiment, the list includes as many objects as unique capture probes. In another embodiment, the list includes the capture probes which interact with the compound. Thus, the list can contain only those capture probes for which an interaction was detected, or only those capture probes for which an interaction met a predetermined condition. Such a list has fewer objects as members than the number of unique capture probes. In a preferred embodiment, the interaction site profile is stored in computer memory, such as random access memory or flash memory, or on computer readable media, such as magnetic (e.g., a diskette, removable hard drive, or internal hard drive) or optical media (e.g., a compact disk (CD), DVD, or holographic media). A profile stored in this manner can be on a personal computer, server, e.g., a network server, or mainframe, and can be accessed from another device across a network, e.g., an intranet or internet. In another embodiment, the interaction site profile is printed on to a media such as a plastic, a paper or a label, e.g., as a bar code or variation thereof.

The value associated with each object of an interaction site profile can be obtained from a quantitative observation, or a qualitative observation, preferably a quantitative observation. In one embodiment, the associated value is a function of the amount of interaction between the compound and the probe. For example, the amount of interaction can be the amount of binding, the amount of probe modification, or affinity. In a preferred embodiment, the associated value is a function of the amount of binding between the compound and the probe. The value can be a function of the amount of a quantitative observation such as a fluorescent signal, a radioactive signal, or a phosphorescent signal of a contacted capture probe. The value can be provided by an instrument, e.g., a CCD camera. In one embodiment, the value is a function of the surface plasmon resonance at the site of a contacted capture probe. In a preferred embodiment, the associated values are adjusted for a background signal. In another embodiment, the associated value is a function of moles of bound compound. In yet another embodiment, the associated value is an affinity, relative affinity, apparent affinity, association constant, dissociation constant, logarithm of an affinity, or free energy for binding, of the compound for the capture probe. In a preferred embodiment, the associated values in the list are differ. In other words, the list contains more than one object, and i.e., the associated values of the objects in the list are not all the same. The values provide a range. The values can be distributed in the range. In some embodiments, the values can approximate a Poisson distribution. The list can contain objects whose associated values are zero, or null. The list can contain objects whose associated values are positive or negative. In one embodiment, the list does not contain any objects whose associated values are zero or null.

In a preferred embodiment, interaction site profiles are provided for a compound at varying concentrations of the compound, i.e. an interaction site profile is provided for a compound at a first concentration, at a second concentration, etc. In another preferred embodiment, interaction site profiles are provided for a compound for interaction with varying concentration of capture probes. For example, an array can have more than one unit, the compositions of the units being identical, but the first unit having the probes at a first concentration, and the second unit having the probes at a second concentration, etc. In yet another preferred embodiment, interaction site profiles are provide for a compound for various intervals after contacting the compound to the array. For example, a first profile can be provided after a first interval of time has elapsed after application, and a second profile can be provided after a second interval, etc.

The capture probes contain a unique region. In one embodiment, the unique region is an interaction site, an interaction site variant, or putative interaction site. In another embodiment, the unique region is random.

The array of the present invention can contain at least two, four, preferably 16, 64, 96, 128, 384, 1536, or more unique capture probes. In one embodiment, the array is a solid silica support. The plurality of nucleic acid probes can be stably attached to the support by a covalent bond. For example, a probe can be stably attached with a silane compound reactive with primary amines, or acrylamide moieties in the oligonucleotide or PCR productions, or an amino silane or polylysine or other polymer capable of UV crosslinking to single-stranded or double-stranded DNA. In a preferred embodiment, the array is a glass slide. The slide can be treated with an activating agent, e.g., 1,4-diphenylene-diisothiocyanate (PDC).

In one embodiment, the capture probes are small organic chemicals, e.g., compounds with a molecular weight of 10,000, 5,000, 3,000, or 1,000 Daltons or less. The chemicals can be produced by combinatorial synthesis.

The capture probes are polypeptides. The polypeptides can contain 2, 10, 20, 30, 50, or 100 or more amino acids. In a preferred embodiment, the polypeptides are antibodies. In a preferred embodiment, the capture probes are nucleic acids. The capture probes can be a deoxyribonucleic acid (DNA), a ribonucleic acid (RNA), a peptide nucleic acid (PNA), or any combination thereof. For example the capture probes can include a DNA, part of which is hybridized to part of an RNA. The capture probes can include a DNA ligated to an RNA or a nucleic acid chemically synthesized to include ribonucleotides and deoxyribonucleotides. In one embodiment, the capture probes are RNA. In a preferred embodiment, the capture probes are DNA, e.g., single-stranded DNA and double-stranded DNA.

In a much preferred embodiment, the capture probes are double-stranded DNA, or consist substantially of double-stranded DNA. The capture probes can be at least about six, ten, 15, 20, 25, 30, 35, 40, 45, 50, 60, 70, 80, 90 or 100 or more basepairs in length. Preferably, the capture probes are about 15 to 90, or about 25 to 50 basepairs in length. In a preferred embodiment, the capture probes are about 15 to 30 basepairs in length. The unique region can comprise two, three, four, five, six, seven, eight, nine, ten, 15, 20, or more basepairs. The unique region can be contiguous, or can be interrupted by one or more common regions. In one embodiment, the DNA capture probes contain fragments of genomic DNA, preferably non-coding regions of genomic DNA, more preferably regulatory regions of genomic DNA, most preferably promoters and enhancers.

In embodiments in which the monitored interaction between the compound and the capture probes is a binding interaction, the unique region is preferably a binding site. In one such embodiment, the array includes all possible combinations of natural basepair substitutions (e.g., adenine-thymidine, thymidine-adenine, guanine-cytosine, cytosine-guanine basepairs) at greater than two basepairs of the interaction site. Thus, the array includes at least 64 unique capture probes. In another such embodiment, the array includes all possible combinations of natural basepair substitutions at greater than three basepairs of the interaction site, i.e. 256 unique capture probes.

The compound for which the interaction site profile is generated can be a polypeptide, a peptoid, a PNA, or a chemically modified polypeptide. In a much preferred embodiment the compound is a polypeptide. The polypeptide can be a nucleic acid binding protein. In one embodiment, the polypeptide is an RNA contacting protein such as a splicing factor, a ribosomal protein, a viral protein, an RNA modification enzyme, a translation factor, and the like. In a preferred embodiment, the polypeptide is a DNA contacting protein such as a transcription factor, a replication factor, a telomere binding protein, a centromere binding protein, a restriction modification enzyme, a DNA methylase, DNA repair protein, a single-stranded DNA binding protein, a recombination protein and the like. In a much preferred embodiment, the polypeptide is a transcription factor. The transcription factor can bind a double stranded DNA sequence with an affinity of 10 mM, 1 mM, 100 nM or less, preferably 10 nM or less, 1 nM or less, and even more preferably 100 pM or less. The transcription factor can be selected from the group consisting of homeodomains, helix-tum-helix motif proteins, beta-sheets, leucine zippers, steroid receptors, zinc fingers and histones. In one embodiment, the polypeptide is a zinc finger polypeptide. In another embodiment, the polypeptide is modified or combined with natural and exotic chemical ligands. In yet another embodiment, the compound for which the interaction site profile is generated includes more than one polypeptide, i.e., polypeptides are combined.

In a preferred embodiment, the polypeptide is covalently attached to bacteriophage, e.g., a T7 phage, a lambdoid phage, or a filamentous phage. Preferably, the polypeptide is covalent attached to a filamentous phage such as fd or M13. The polypeptide can be covalently fused to a coat protein by constructing a fusion gene with the gene encoding the polypeptide an the viral coat protein gene, e.g., filamentous phage gene VIII or gene III. In another preferred embodiment, the polypeptide is covalently attached to green fluorescent protein (GFP), or a variant thereof (such as enhanced GPP, CFP, BFP, and the like). The polypeptide can be covalently attached by constructing a fusion gene. In yet another embodiment, the polypeptide is linked with an unrelated sequence, e.g., a fusion protein, purification handle, or epitope tag. Useful examples of such unrelated sequences include maltose binding protein, glutathione-S-transferase, chitin binding protein, thioredoxin, hexa-histidine (or 6-His), the “FLAG tag”, the myc epitope, and the hemagglutinin epitope.

In one embodiment, the polypeptide contains a detectable label. The detectable label can be a radiolabel. Preferably, the detectably label is a fluorescent label, e.g., malachite green, Oregon green, Texas Red, Congo Red, Cy3, SybrGreen I, or R-phycoerythrin. In another embodiment, the polypeptide is contacted with an antibody. The antibody can contact the protein directly or can contact a covalently attached tag, e.g., a moiety mentioned above.

In a preferred embodiment, the polypeptide is a variant of a natural counterpart. The variant can have at least one amino acid difference from the natural counterpart. Preferably the differing amino acid is located within 50 Ångstroms, 20 Ångstroms, or 10 Ångstroms or less of the bound nucleic acid in a structural model, e.g., a model built from X-ray diffraction data, NMR restraint data, or another homology model.

The predetermined criterion can be a desired affinity for interaction with a nucleic acid (e.g., a desired DNA binding site specificity.) In one embodiment, the predetermined criterion is a binding affinity for a selected target compound, the compound being one of the capture probes. In another embodiment, the predetermined criterion is a first predetermined affinity for a first target and a second predefined affinity for a second target, etc. In yet another embodiment, the predetermined criterion is itself a interaction site profile, e.g., one designed for a specific application.

The plurality of polypeptides can be proteins from a provided sample, from a patient sample, or from a cell lysate. In a preferred embodiment, the plurality of polypeptides is from an expression library, e.g., a genomic or cDNA library, or a specialized library of nucleic acid binding proteins. In a much preferred embodiment, the plurality of polypeptides are variants of a progenitor polypeptide. The variants can be generated by a method including cassette mutagenesis, PCR mutagenesis, and the like. In a preferred embodiment, the variants have amino acid differences from the progenitor in at least one amino acid that is within 10 Ångstroms of the nucleic acid binding interface, e.g., an interface identified by a structural model or by mutagenesis. In a preferred embodiment, such variants are in a transcription factor.

In a preferred embodiment, the interaction site profiles of each member of the plurality are determined individually. In another embodiment, the individual members of the plurality are pooled, and an interaction site profile of the pool is identified. If the profile of the pool meets the predetermined criterion, the pool can be subdivided or split such that interaction site profiles are identified for smaller pools or for individual members of the pool. The process can be repeated until an individual polypeptide is selected.

In another aspect, the current invention features a polypeptide selected by the method of selecting a polypeptide which meets a predetermined criterion. The method includes: providing a plurality of polypeptides, identifying the interaction site profile of each polypeptide variant of the plurality by the methods described above; selecting the polypeptide whose interaction site profile meets the predetermined criterion to thereby select a polypeptide which meets a predetermined criterion.

In another aspect, the invention features a method of designing a polypeptide to bind a desired DNA binding site. The method includes providing a reference protein with at least two domains that contact DNA; providing a plurality of variants in each domain, the variants being different from a reference domain by at least one amino acid in the interface which contacts DNA; determining the interaction site profiles for each of the plurality of variants a method described herein; selecting, for each domain, variants whose interaction site profile manifests specificity for a fragment of the desired DNA binding site; linking selected variants of each domain to provide at least one candidate polypeptide; determining the interaction site profiles for each candidate polypeptide by a method described herein; selecting candidate polypeptides whose interaction site profile indicates specificity for a desired DNA binding site to thereby select a polypeptide with a desired DNA binding site specificity.

In one embodiment, the interaction of the compound with the probe results in a covalent modification of the probe, e.g., a covalent bond of the probe can be broken or formed. In a preferred embodiment, the interaction of the compound with the capture probe is a binding interaction wherein neither the compound nor the probe has a covalent bond broken or formed.

In a preferred embodiment, the interaction site profile is a list of objects, each object representing a unique capture probe, and having an associated value, preferably a numerical value. The list can contain two, three, four, five, six, seven, eight, nine, ten, 15, 20, 50, 100, 1000 or more objects. In a preferred embodiment, each unique capture probe is represented by an object. In this embodiment, the list includes as many objects as unique capture probes. In another embodiment, the list includes the capture probes which interact with the compound. Thus, the list can contain only those capture probes for which an interaction was detected, or only those capture probes for which an interaction met a predetermined condition. Such a list has fewer objects as members than the number of unique capture probes. In a preferred embodiment, the interaction site profile is stored in computer memory, such as random access memory or flash memory, or on computer readable media, such as magnetic (e.g., a diskette, removable hard drive, or internal hard drive) or optical media (e.g., a compact disk (CD), DVD, or holographic media). A profile stored in this manner can be on a personal computer, server, e.g., a network server, or mainframe, and can be accessed from another device across a network, e.g., an intranet or internet. In another embodiment, the interaction site profile is printed on to a media such as a plastic, a paper or a label, e.g., as a bar code or variation thereof.

The value associated with each object of an interaction site profile can be obtained from a quantitative observation, or a qualitative observation, preferably a quantitative observation. In one embodiment, the associated value is a function of the amount of interaction between the compound and the probe. For example, the amount of interaction can be the amount of binding, the amount of probe modification, or affinity. In a preferred embodiment, the associated value is a function of the amount of binding between the compound and the probe. The value can be a function of the amount of a quantitative observation such as a fluorescent signal, a radioactive signal, or a phosphorescent signal of a contacted capture probe. The value can be provided by an instrument, e.g., a CCD camera. In one embodiment, the value is a function of the surface plasmon resonance at the site of a contacted capture probe. In a preferred embodiment, the associated values are adjusted for a background signal. In another embodiment, the associated value is a function of moles of bound compound. In yet another embodiment, the associated value is an affinity, relative affinity, apparent affinity, association constant, dissociation constant, logarithm of an affinity, or free energy for binding, of the compound for the capture probe. In a preferred embodiment, the associated values in the list are differ. In other words, the list contains more than one object, and i.e., the associated values of the objects in the list are not all the same. The values provide a range. The values can be distributed in the range. In some embodiments, the values can approximate a Poisson distribution. The list can contain objects whose associated values are zero, or null. The list can contain objects whose associated values are positive or negative. In one embodiment, the list does not contain any objects whose associated values are zero or null.

In a preferred embodiment, interaction site profiles are provided for a compound at varying concentrations of the compound, i.e. an interaction site profile is provided for a compound at a first concentration, at a second concentration, etc. In another preferred embodiment, interaction site profiles are provided for a compound for interaction with varying concentration of capture probes. For example, an array can have more than one unit, the compositions of the units being identical, but the first unit having the probes at a first concentration, and the second unit having the probes at a second concentration, etc. In yet another preferred embodiment, interaction site profiles are provide for a compound for various intervals after contacting the compound to the array. For example, a first profile can be provided after a first interval of time has elapsed after application, and a second profile can be provided after a second interval, etc.

The capture probes contain a unique region. In one embodiment, the unique region is an interaction site, an interaction site variant, or putative interaction site. In another embodiment, the unique region is random.

The array of the present invention can contain at least two, four, preferably 16, 64, 96, 128, 384, 1536, or more unique capture probes. In one embodiment, the array is a solid silica support. The plurality of nucleic acid probes can be stably attached to the support by a covalent bond. For example, a probe can be stably attached with a silane compound reactive with primary amines, or acrylamide moieties in the oligonucleotide or PCR productions, or an amino silane or polylysine or other polymer capable of UV crosslinking to single-stranded or double-stranded DNA. In a preferred embodiment, the array is a glass slide. The slide can be treated with an activating agent, e.g., 1,4-diphenylene-diisothiocyanate (PDC).

In one embodiment, the capture probes are small organic chemicals, e.g., compounds with a molecular weight of 10,000, 5,000, 3,000, or 1,000 Daltons or less. The chemicals can be produced by combinatorial synthesis.

The capture probes are polypeptides. The polypeptides can contain 2, 10, 20, 30, 50, or 100 or more amino acids. In a preferred embodiment, the polypeptides are antibodies. In a preferred embodiment, the capture probes are nucleic acids. The capture probes can be a deoxyribonucleic acid (DNA), a ribonucleic acid (RNA), a peptide nucleic acid (PNA), or any combination thereof. For example the capture probes can include a DNA, part of which is hybridized to part of an RNA. The capture probes can include a DNA ligated to an RNA or a nucleic acid chemically synthesized to include ribonucleotides and deoxyribonucleotides. In one embodiment, the capture probes are RNA. In a preferred embodiment, the capture probes are DNA, e.g., single-stranded DNA and double-stranded DNA.

In a much preferred embodiment, the capture probes are double-stranded DNA, or consist substantially of double-stranded DNA. The capture probes can be at least about six, ten, 15, 20, 25, 30, 35, 40, 45, 50, 60, 70, 80, 90 or 100 or more basepairs in length. Preferably, the capture probes are about 15 to 90, or about 25 to 50 basepairs in length. In a preferred embodiment, the capture probes are about 15 to 30 basepairs in length. The unique region can comprise two, three, four, five, six, seven, eight, nine, ten, 15, 20, or more basepairs. The unique region can be contiguous, or can be interrupted by one or more common regions. In one embodiment, the DNA capture probes contain fragments of genomic DNA, preferably non-coding regions of genomic DNA, more preferably regulatory regions of genomic DNA, most preferably promoters and enhancers.

In embodiments in which the monitored interaction between the compound and the capture probes is a binding interaction, the unique region is preferably a binding site. In one such embodiment, the array includes all possible combinations of natural basepair substitutions (e.g., adenine-thymidine, thymidine-adenine, guanine-cytosine, cytosine-guanine basepairs) at greater than two basepairs of the interaction site. Thus, the array includes at least 64 unique capture probes. In another such embodiment, the array includes all possible combinations of natural basepair substitutions at greater than three basepairs of the interaction site, i.e. 256 unique capture probes.

The compound for which the interaction site profile is generated can be a polypeptide, a peptoid, a PNA, or a chemically modified polypeptide. In a much preferred embodiment the compound is a polypeptide. The polypeptide can be a nucleic acid binding protein. In one embodiment, the polypeptide is an RNA contacting protein such as a splicing factor, a ribosomal protein, a viral protein, an RNA modification enzyme, a translation factor, and the like. In a preferred embodiment, the polypeptide is a DNA contacting protein such as a transcription factor, a replication factor, a telomere binding protein, a centromere binding protein, a restriction modification enzyme, a DNA methylase, DNA repair protein, a single-stranded DNA binding protein, a recombination protein and the like. In a much preferred embodiment, the polypeptide is a transcription factor. The transcription factor can bind a double stranded DNA sequence with an affinity of 10 mM, 1 mM, 100 nM or less, preferably 10 nM or less, 1 nM or less, and even more preferably 100 pM or less. The transcription factor can be selected from the group consisting of homeodomains, helix-turn-helix motif proteins, beta-sheets, leucine zippers, steroid receptors, zinc fingers and histones. In one embodiment, the polypeptide is a zinc finger polypeptide. In another embodiment, the polypeptide is modified or combined with natural and exotic chemical ligands. In yet another embodiment, the compound for which the interaction site profile is generated includes more than one polypeptide, i.e., polypeptides are combined.

In a preferred embodiment, the polypeptide is covalently attached to bacteriophage, e.g., a T7 phage, a lambdoid phage, or a filamentous phage. Preferably, the polypeptide is covalent attached to a filamentous phage such as fd or M13. The polypeptide can be covalently fused to a coat protein by constructing a fusion gene with the gene encoding the polypeptide an the viral coat protein gene, e.g., filamentous phage gene VIII or gene III. In another preferred embodiment, the polypeptide is covalently attached to green fluorescent protein (GFP), or a variant thereof (such as enhanced GFP, CFP, BFP, and the like). The polypeptide can be covalently attached by constructing a fusion gene. In yet another embodiment, the polypeptide is linked with an unrelated sequence, e.g., a fusion protein, purification handle, or epitope tag. Useful examples of such unrelated sequences include maltose binding protein, glutathione-S-transferase, chitin binding protein, thioredoxin, hexa-histidine (or 6-His), the “FLAG tag”, the myc epitope, and the hemagglutinin epitope.

In one embodiment, the polypeptide contains a detectable label. The detectable label can be a radiolabel. Preferably, the detectably label is a fluorescent label, e.g., malachite green, Oregon green, Texas Red, Congo Red, Cy3, SybrGreen I, or R-phycoerythrin. In another embodiment, the polypeptide is contacted with an antibody. The antibody can contact the protein directly or can contact a covalently attached tag, e.g., a moiety mentioned above.

In a preferred embodiment, the polypeptide is a variant of a natural counterpart. The variant can have at least one amino acid difference from the natural counterpart. Preferably the differing amino acid is located within 50 Ångstroms, 20 Ångstroms, or 10 Ångstroms or less of the bound nucleic acid in a structural model, e.g., a model built from X-ray diffraction data, NMR restraint data, or another homology model.

In another aspect, the invention features evaluating a plurality of polypeptides. The method includes: providing a plurality of polypeptide variants; providing an array of a plurality of nucleic acid probes, wherein each of the probes in the plurality is positionally distinguishable from other probes of the plurality, and wherein each positionally distinguishable probe includes a unique region which corresponds to a binding site for the polypeptide; contacting the plurality of polypeptides with the array of probes; identifying probes to which the plurality of polypeptides interacts thereby identifying an interaction site profile; assessing the interaction site profile for interactions for desired nucleic acid sequences to thereby evaluate the plurality of polypeptides.

The plurality of polypeptides can be proteins from a provided sample, from a patient sample, or from a cell lysate. In a preferred embodiment, the plurality of polypeptides is from an expression library, e.g., a genomic or cDNA library, or a specialized library of nucleic acid binding proteins. In a much preferred embodiment, the plurality of polypeptides are variants of a progenitor polypeptide. The variants can be generated by a method including cassette mutagenesis, PCR mutagenesis, and the like. In a preferred embodiment, the variants have amino acid differences from the progenitor in at least one amino acid that is within 10 Ångstroms of the nucleic acid binding interface, e.g., an interface identified by a structural model or by mutagenesis. In a preferred embodiment, such variants are in a transcription factor.

In another aspect, the invention features a method of screening a nucleic acid sequence for the presence of candidate sites for a compound. The method includes: providing a interaction site profile for a compound, by a method described herein; providing a nucleic acid sequence; selecting interaction sites from the interaction site profile, the selected interaction sites having an associated value that meets a preselected requirement; indicating the presence or absence of the selected interaction sites in the nucleic acid sequence to thereby screen a nucleic acid sequence for candidate sites for interaction with a compound.

In one embodiment, the interaction of the compound with the probe results in a covalent modification of the probe, e.g., a covalent bond of the probe can be broken or formed. In a preferred embodiment, the interaction of the compound with the capture probe is a binding interaction wherein neither the compound nor the probe has a covalent bond broken or formed.

In a preferred embodiment, the interaction site profile is a list of objects, each object representing a unique capture probe, and having an associated value, preferably a numerical value. The list can contain two, three, four, five, six, seven, eight, nine, ten, 15, 20, 50, 100, 1000 or more objects. In a preferred embodiment, each unique capture probe is represented by an object. In this embodiment, the list includes as many objects as unique capture probes. In another embodiment, the list includes the capture probes which interact with the compound. Thus, the list can contain only those capture probes for which an interaction was detected, or only those capture probes for which an interaction met a predetermined condition. Such a list has fewer objects as members than the number of unique capture probes. In a preferred embodiment, the interaction site profile is stored in computer memory, such as random access memory or flash memory, or on computer readable media, such as magnetic (e.g., a diskette, removable hard drive, or internal hard drive) or optical media (e.g., a compact disk (CD), DVD, or holographic media). A profile stored in this manner can be on a personal computer, server, e.g., a network server, or mainframe, and can be accessed from another device across a network, e.g., an intranet or internet. In another embodiment, the interaction site profile is printed on to a media such as a plastic, a paper or a label, e.g., as a bar code or variation thereof.

The value associated with each object of an interaction site profile can be obtained from a quantitative observation, or a qualitative observation, preferably a quantitative observation. In one embodiment, the associated value is a function of the amount of interaction between the compound and the probe. For example, the amount of interaction can be the amount of binding, the amount of probe modification, or affinity. In a preferred embodiment, the associated value is a function of the amount of binding between the compound and the probe. The value can be a function of the amount of a quantitative observation such as a fluorescent signal, a radioactive signal, or a phosphorescent signal of a contacted capture probe. The value can be provided by an instrument, e.g., a CCD camera. In one embodiment, the value is a function of the surface plasmon resonance at the site of a contacted capture probe. In a preferred embodiment, the associated values are adjusted for a background signal. In another embodiment, the associated value is a function of moles of bound compound. In yet another embodiment, the associated value is an affinity, relative affinity, apparent affinity, association constant, dissociation constant, logarithm of an affinity, or free energy for binding, of the compound for the capture probe. In a preferred embodiment, the associated values in the list are differ. In other words, the list contains more than one object, and i.e., the associated values of the objects in the list are not all the same. The values provide a range. The values can be distributed in the range. In some embodiments, the values can approximate a Poisson distribution. The list can contain objects whose associated values are zero, or null. The list can contain objects whose associated values are positive or negative. In one embodiment, the list does not contain any objects whose associated values are zero or null.

In a preferred embodiment, interaction site profiles are provided for a compound at varying concentrations of the compound, i.e. an interaction site profile is provided for a compound at a first concentration, at a second concentration, etc. In another preferred embodiment, interaction site profiles are provided for a compound for interaction with varying concentration of capture probes. For example, an-array can have more than one unit, the compositions of the units being identical, but the first unit having the probes at a first concentration, and the second unit having the probes at a second concentration, etc. In yet another preferred embodiment, interaction site profiles are provide for a compound for various intervals after contacting the compound to the array. For example, a first profile can be provided after a first interval of time has elapsed after application, and a second profile can be provided after a second interval, etc.

The capture probes contain a unique region. In one embodiment, the unique region is an interaction site, an interaction site variant or putative interaction site. In another embodiment, the unique region is random.

The array of the present invention can contain at least two, four, preferably 16, 64, 96, 128,384, 1536, or more unique capture probes. In one embodiment, the array is a solid silica support. The plurality of nucleic acid probes can be stably attached to the support by a covalent bond. For example, a probe can be stably attached with a silane compound reactive with primary amines, or acrylamide moieties in the oligonucleotide or PCR productions, or an amino silane or polylysine or other polymer capable of UV crosslinking to single-stranded or double-stranded DNA. In a preferred embodiment, the array is a glass slide. The slide can be treated with an activating agent, e.g., 1,4-diphenylene-diisothiocyanate (PDC).

In one embodiment, the capture probes are small organic chemicals, e.g., compounds with a molecular weight of 10,000, 5,000,3,000, or 1,000 Daltons or less. The chemicals can be produced by combinatorial synthesis. The capture probes are polypeptides. The polypeptides can contain 2, 10, 20, 30, 50, or 100 or more amino acids. In a preferred embodiment, the polypeptides are antibodies. In a preferred embodiment, the capture probes are nucleic acids. The capture probes can be a deoxyribonucleic acid ([DNA), a ribonucleic acid (RNA), a peptide nucleic acid (PNA), or any combination thereof. For example the capture probes can include a DNA, part of which is hybridized to part of an RNA. The capture probes can include a DNA ligated to an RNA or a nucleic acid chemically synthesized to include ribonucleotides and deoxyribonucleotides. In one embodiment, the capture probes are RNA. In a preferred embodiment, the capture probes are DNA, e.g., single-stranded DNA and double-stranded DNA.

In a much preferred embodiment, the capture probes are double-stranded DNA, or consist substantially of double-stranded DNA. The capture probes can be at least about six, ten, 15, 20, 25, 30, 35, 40, 45, 50, 60, 70, 80, 90 or 100 or more basepairs in length. Preferably, the capture probes are about 15 to 90, or about 25 to 50 basepairs in length. In a preferred embodiment, the capture probes are about 15 to 30 basepairs in length. The unique region can comprise two, three, four, five, six, seven, eight, nine, ten, 15, 20, or more basepairs. The unique region can be contiguous, or can be interrupted by one or more common regions. In one embodiment, the DNA capture probes contain fragments of genomic DNA, preferably non-coding regions of genomic DNA, more preferably regulatory regions of genomic DNA, most preferably promoters and enhancers.

In embodiments in which the monitored interaction between the compound and the capture probes is a binding interaction, the unique region is preferably a binding site. In one such embodiment, the array includes all possible combinations of natural basepair substitutions (e.g., adenine-thymidine, thymidine-adenine, guanine-cytosine, cytosine-guanine basepairs) at greater than two basepairs of the interaction site. Thus, the array includes at least 64 unique capture probes. In another such embodiment, the array includes all possible combinations of natural basepair substitutions at greater than three basepairs of the interaction site, i.e. 256 unique capture probes.

The compound for which the interaction site profile is generated can be a polypeptide, a peptoid, a PNA, or a chemically modified polypeptide. In a much preferred embodiment the compound is a polypeptide. The polypeptide can be a nucleic acid binding protein. In one embodiment, the polypeptide is an RNA contacting protein such as a splicing factor, a ribosomal protein, a viral protein, an RNA modification enzyme, a translation factor, and the like. In a preferred embodiment, the polypeptide is a DNA contacting protein such as a transcription factor, a replication factor, a telomere binding protein, a centromere binding protein, a restriction modification enzyme, a DNA methylase, DNA repair protein, a single-stranded DNA binding protein, a recombination protein and the like. In a much preferred embodiment, the polypeptide is a transcription factor. The transcription factor can bind a double stranded DNA sequence with an affinity of 10 mM, 1 mM, 100 nM or less, preferably 10 nM or less, 1 nM or less, and even more preferably 100 pM or less. The transcription factor can be selected from the group consisting of homeodomains, helix-turn-helix motif proteins, beta-sheets, leucine zippers, steroid receptors, zinc fingers and histones. In one embodiment, the polypeptide is a zinc finger polypeptide. In another embodiment, the polypeptide is modified or combined with natural and exotic chemical ligands. In yet another embodiment, the compound for which the interaction site profile is generated includes more than one polypeptide, i.e., polypeptides are combined.

In a preferred embodiment, the polypeptide is covalently attached to bacteriophage, e.g., a T7 phage, a lambdoid phage, or a filamentous phage. Preferably, the polypeptide is covalent attached to a filamentous phage such as fd or M13. The polypeptide can be covalently fused to a coat protein by constructing a fusion gene with the gene encoding the polypeptide an the viral coat protein gene, e.g., filamentous phage gene VIII or gene III. In another preferred embodiment, the polypeptide is covalently attached to green fluorescent protein (GFP), or a variant thereof (such as enhanced GFP, CFP, BFP, and the like). The polypeptide can be covalently attached by constructing a fusion gene. In yet another embodiment, the polypeptide is linked with an unrelated sequence, e.g., a fusion protein, purification handle, or epitope tag. Useful examples of such unrelated sequences include maltose binding protein, glutathione-S-transferase, chitin binding protein, thioredoxin, hexa-histidine (or 6-His), the “FLAG tag”, the myc epitope, and the hemagglutinin epitope.

In one embodiment, the polypeptide contains a detectable label. The detectable label can be a radiolabel. Preferably, the detectably label is a fluorescent label, e.g., malachite green, Oregon green, Texas Red, Congo Red, Cy3, SybrGreen I, or R-phycoerythrin. In another embodiment, the polypeptide is contacted with an antibody. The antibody can contact the protein directly or can contact a covalently attached tag, e.g., a moiety mentioned above.

In a preferred embodiment, the polypeptide is a variant of a natural counterpart. The variant can have at least one amino acid difference from the natural counterpart. Preferably the differing amino acid is located within 50 Ångstroms, 20 Ångstroms, or 10 Ångstroms or less of the bound nucleic acid in a structural model, e.g., a model built from X-ray diffraction data, NMR restraint data, or another homology model.

In a preferred embodiment, the interaction site profile and the nucleic acid sequence are stored in computer memory and/or on computer readable medium.

In one embodiment, the nucleic acid sequence is an episome or plasmid. In a preferred embodiment, the nucleic acid sequence is genomic nucleic acid sequence or a fragment thereof. The nucleic acid sequence can include a contig, a chromosome, a genome, or a database.

In a preferred embodiment, the nucleic acid sequence largely consists of non-coding regions of genomic DNA, preferably regulatory regions of genomic DNA, most preferably promoters and enhancers. In another preferred embodiment, the nucleic acid sequence is a database, e.g., an external database such as GenBank.

In another aspect, the invention features a database of interaction sites. The database includes more than one record, and at least one record referencing a nucleic acid sequence which is a candidate site identified a method described herein. In one embodiment, the database references a hit to an external database, the hit being identified as a candidate site by a method described herein.

In another aspect, the invention features a computer program product comprising a computer-useable medium having a computer-readable program code embodied on it. The program code instructs a computer system to receive as input an interaction site profile; to access a database of nucleic acid sequence; to identify candidate sites in the database by a method described herein; and to output candidate sites in the database. The interaction site profile can be received as input from a user, from an instrument, from a database, such as one described herein, or from another computer. The database of nucleic acid sequence can be accessed from another computer, e.g., across a computer network, from computer memory, or from computer readable medium. In one embodiment, the database is an external database, e.g., GenBank. In another embodiment, the database contains selected nucleic acid sequences. The database can include sequences from a species, e.g., maize, rice, Arabidopsis, E. coli , B. subtilis, Drosophila, Saccharomyces cerevisiae, mouse, or human. The database can include sequences implicated in a disease or disease, e.g., a set nucleic acid sequences regulating genes overexpressed in cancer cells, a set nucleic acid sequences related to human genetic disorders, a set of nucleic acid sequences implicated in hypertension, stroke, metastasis, obesity, and the like.

In another aspect, the current invention features a database that includes records that contain a reference to a compound and a reference to the interaction site profile of the compound. The interaction site profile is provided by a method described herein. The database can be stored on computer readable media or in computer memory. In a preferred embodiment, the database is stored in computer memory, such as random access memory or flash memory, or on computer readable media, such as magnetic (e.g., a diskette, removable hard drive, or internal hard drive) or optical media (e.g., a compact disk (CD), DVD, or holographic media). A database stored in this manner can be on a personal computer, server, e.g., a network server, or mainframe, and can be accessed from another device across a network, e.g., an intranet or internet. In one embodiment, the database is stored on one computer and is accessed across a network by another computer. The database can be accessed by a user interface which accepts queries for a compound or group of compounds, and returns one or more interaction site profiles. Alternatively, the interface can accept an interaction site profile and return a compound or group of compounds by identifying in the database interactions site profiles that-meet a preselected condition of similarity to the input profile. In a preferred embodiment, the interface also accepts parameters to specify the preselected condition.

In another aspect, the current invention features a method of predicting the sites bound by a compound in a genome or fragments. The method includes evaluating the level of active compound molecules in a cell; obtaining the interaction site profile of the compound by a method described herein; determining the number of occurrences in the nucleic acid sequences of the genome or fragments thereof for each of the plurality of sites in the interaction profile to thereby predict the interactions sites bound by a compound in a cell. In one embodiment, a probability is determined for each of the plurality of interaction sites such that the probably represents that likelihood that a compound is interacting with the site.

In another aspect, the current inventions features a method of identifying a regulatory protein for a plurality of coregulated genes. The method includes: providing a regulatory nucleic acid sequence for each member of the plurality; providing a set of interaction site profiles for a set of reference proteins; identifying for each reference protein candidate interaction sites within the regulatory nucleic acid sequence of each member of the plurality of coregulated genes by a method described herein; selecting the reference proteins that have candidate interaction sites for a number of coregulated genes, the number being greater than a threshold value to thereby identify a regulatory protein for a plurality of coregulated genes. In one embodiment, the set of interaction site profiles is provided by a database described herein.

As used herein, the term “polypeptide” is defined to encompass a polymer of two or more amino acids joined by a peptide bond. It include full length proteins, e.g., recombinant proteins and proteins isolated from natural sources. Thus, a “polypeptide” refers to a dipeptide, a tripeptide, peptides of four, five, six, seven, eight, nine, ten or more amino acids, as well as peptides of at least 10, 20, 30, 40, 50, 75, 100, 150, 200 or more amino acids. Both “polypeptides” and “proteins” include species which are further modified, e.g., by phosphorylation, glycosylation, methylation, acylation, and the like.

As used herein the term “interaction site” is a site at which a molecule, e.g., a polypeptide, e.g., a protein, physically interacts, e.g., binds to or modifies, a second molecule, e.g., a substrate, a nucleic acid binding site, or a polypeptide.

As used herein with regard to individual nucleic acid sequences, the term “randomized” refers to in vitro-synthesized sequences in which any nucleotide or ribonucleotide can be present at one, more than one or all positions; therefore, for such positions as are randomized, the sequence of the finished molecule is not pre-determined, but is left to chance.

As used herein with regard to an array of the invention, the term “randomized” refers to an array which is constructed such that, for a sequence of a recognition site within a nucleic acid sequence-of a protein of a selected length (e.g. a hexamer), each possible nucleotide combination is comprised by a corresponding feature thereof. In order to realize a complete set of such nucleotide sequence permutations, it is necessary to specify fully the sequence of each feature during synthesis of the array; therefore, while such an array may be referred to as an “array of randomized 6-mers” the design of the array is entirely non-random.

As used herein, the term “half-site” refers to a nucleic acid sequence which is recognized and bound by a targeting amino acid sequence present on one protein subunit of a dimeric protein complex. Neither subunit of the dimeric protein complex will bind its cognate half-site alone (i.e., unless dimerized to the other); therefore, either both half-sites are occupied by protein, or neither is. Both half sites of a recognition site within a nucleic acid sequence for a protein may be identical, whether arranged head-to-tail or as a palindrome (head-to-head or tail-to-tail); if in the latter configuration, the sequence of a recognition site within a nucleic acid sequence of a protein is said to have “dyad symmetry”. Typically, a recognition site within a nucleic acid sequence for a protein bound by a protein homodimer comprises two identical half-sites. Alternatively, the two half-sites comprised by a recognition site within a nucleic acid sequence for a protein may be unlike in sequence; it is usually true that dissimilar half-sites are bound by different targeting amino acid sequences, as would be found on the two subunits of a protein heterodimer. Depending on their orientation relative to one another, recognition sites within a nucleic acid sequence for a protein comprising non-identical, but similar, half-sites may also be said to have dyad symmetry.

It contemplated that a chimeric (or “fusion”) protein according to the invention comprises a protein which binds a recognition site within a nucleic acid sequence for a protein, fused to a second protein component comprising any one of a receptor, an enzyme, a candidate enzyme domain such as a kinase or a protease domain, a candidate protein:protein dimerization domain, a candidate ligand binding domain, or a substrate for a protein-directed enzymatic reaction. In this context, a “protein” is either a whole protein or a protein fragment which retains its ability to recognize- and bind specifically to a recognition site within a nucleic acid sequence for a protein-on a nucleic acid molecule to which site the native, whole protein binds.

As used herein, the term “domain” is a portion of a protein molecule which is sufficient for the performance of a given function, whether in the presence or absence of other sequences of the protein. It is contemplated that a domain is encoded by an uninterrupted amino acid sequence, such that it may be physically cleaved whole away from other amino acid sequence elements and such that it will fold properly without the influence of neighboring sequences.

A “set of co-regulated genes” refers to a number of genes, in the range of about 2 to about 30 genes, that exhibit a given response (in terms of gene expression) to an external stimulus or a given response to a mutation in a specific gene. An example of the latter is where a mutation in the coding region of gene X results in a change in expression levels of genes A-Z. The term “co-regulated set of genes” additionally encompasses genes which are normally under the control of a common trans-regulatory factor, such as a protein. The upper limit on the number in a set of co-regulated genes (i.e., “positives” or up-regulated genes; or “negatives” or down-regulated genes) may be on the order of several thousand.

As used herein, the term “database” refers to at least one table of information, containing at least one record. A record is a row in the table. A record can have one or more fields or attributes. For example, in a database of interaction site profiles, a record can have fields describing the location of a capture probe on an array, the composition, e.g., nucleic acid sequence, of the capture probe at the location, and/or a value, e.g., a numerical value, which is a function of the extent of interaction of the capture probe with a compound. A “sequence database” contains biomolecular sequences, and optionally information associated with the sequences, such as the origin of a sequence (e.g., a library from which the sequence was isolated, the species), the location of a sequence in another database, and/or the position of the sequence in a genome. An external sequence database can include the GenBank database maintained by the National Center for Biotechnology Information (NCBI, http://www.ncbi.nlm.nih.gov/), and the TIGR database maintained by The Institute for Genomic Research (http://www.tigr.org/).

As used herein, the term “library” refers to a physical pool of DNA fragments that is propagated in a cloning vector. For example, the library can contain sequences encoding heterologous polypeptides.

The present invention features a nucleic acid microarray and microarray binding methods which provides high-throughput, sensitive, and quantitative measurements of DNA-protein interactions. The highly parallel nature of these experiments can immediately rank the DNA binding preferences of a protein or pool of proteins.

Also encompassed in this invention are objective metrics of protein specificity which allow highly related proteins to be distinguished by their affinities for sub-optimal DNA sites. These experiments can also distinguish highly related proteins based upon their affinities for sub-optimal DNA sites. Both ranking preferences and distinguishing by sub-optimal affinity were considered in calculating three types of DNA binding specificity scores as metrics of how specific a protein is for its most tightly binding DNA site, as well as of the general spectrum of affinities that the protein has for variant DNA sites (Table 2). These binding specificity scores will allow determination of transcription factors that are likely to bind at only a few specific sequences versus those which are likely to bind more uniformly throughout the genome.

Since dozens of microarray binding experiments could be performed in parallel in a single day, this technology provides significant cost and time advantages over conventional methods such as gel mobility shift assays and nitrocellulose binding assays, which can take months or even years to measure the effects of mutations for a large set of variant DNA-protein interactions. These microarray binding experiments are highly scalable, and thus could readily be adapted for whole genome analyses of transcription factor binding sites. This microarray-based method encourages comprehensive and directly comparable measures that would have been prohibitive in the past, because of laborious experimental procedures.

The materials and instrumentation used in the microarray experiments of the current invention are commercially available and widely used in laboratories employing DNA chips for mRNA expression analysis. In addition, the antibodies used for detection of the bound proteins are commercially available and universal, in that they can be used regardless of what DNA binding domain is displayed on the phage. These experiments are applicable to any polypeptide-nucleic acid interaction in which sequence determinants in both species dictate specificity and affinity.

Identifying small differences in the DNA sequence specificity of distinct transcription factors is highly desirable, since a single nucleotide difference in a DNA binding site can dictate which transcription factor binds at that site. For example, the presence of a T immediately 5′ to the common CACGTG recognition sequence for the yeast basic region/helix-loop-helix proteins PHO4 and CPF-1 inhibits binding of PHO4 (F. Fisher, C. Goding, EMBO J. 11, 4103 (1992)).

Such microarray binding experiments would produce datasets that would be useful not only for predicting functions for previously uncharacterized transcription factors, but also for elucidating regulatory networks. Additionally, the effects of different concentrations of cofactors as well as the effects of alternate cofactors or binding conditions could be measured. This technology will also be immediately useful in engineering designer zinc finger DNA binding domains for the control of gene expression in biotechnology applications ranging from functional genomics to gene therapy. Finally, as more and more of these experiments are performed, the vast datasets produced could yield the necessary data required to determine what rules exist governing DNA recognition by sequence-specific transcription factors.

### DESCRIPTION OF THE DRAWINGS

FIG. 1. Design of microarrays for binding experiments. (A) Model depicting interactions between the Zif268 phage display library and the DNA used in phage selections. The three zinc fingers of Zif268 (F1, F2, and F3) are aligned to show contacts to the nucleotides of the DNA binding site as inferred from the crystal structure of Zif268 and biochemical experiments. The zinc finger amino acid positions are numbered relative to the first helical residue (position 1). The randomized positions in the et-helix of the second finger are circled. DNA base pairs marked X were fixed as given sequences and used to select sequence-specific zinc finger phage from the library. (B) Design of the DNA sequences spotted on the microarrays used in the microarray binding experiments. F1, F2, and F3 refer to fingers 1, 2, and 3 of Zif268 variants, and the dashed boxes indicate the three corresponding triplet binding sites for the three fingers. DNA base pairs marked X were systematically varied to explore the 64 different triplet binding sites for finger 2. The diagram shows attachment of the DNA to a glass slide via an amino linker. (C) Entire microarray, showing all nine replicates, bound by wildtype Zif268 phage. The fluorescent signal intensities of the spots are shown, corresponding to the DNA binding affinities of the protein for the different DNA sequences. The Cy3-labeled alignment oligonucleotide was spotted above and below each column, as well as to the right and left of each row, along the perimeter of the nine replicates. In addition, four spots of DNA containing the wildtype Zif268 binding site were spotted at higher concentrations in each of the four comers (row 1, columns 1-4; rows 1-4, column 10; row 8, columns 7-10; rows 5-8, column 1) of each replicate, as a positive control for wildtype phage binding to the microarrays in preliminary experiments. (I)) Amino acid sequences of the variant a-helical regions in finger 2 of the Zif268 variants used in this study. The randomized positions are marked with an X. The three primary recognition positions are highlighted. The names of the clones are listed to the left of these sequences. The first variant listed is wildtype Zif268.

FIG. 2. Wildtype and variant Zif268 zinc finger phage bound to microarrays. One of nine replicates on each microarray slide is shown for each of the binding experiments described. Spots with high relative signal intensities for each of the Zif268 variants labeled in descending numerical order according to decreasing K_{d}^{app} values, and the sequences corresponding to each of these numbered spots are listed between the microarray images and the sequence logos. The fluorescent signal intensities of the spots are shown, corresponding to the DNA binding affinities. The bars were calibrated from the apparent binding constants (K^{app}'S) determined using ELISA. Sequence logos depicting the DNA binding site preferences of the variant zinc fingers are shown to the right of the microarrays. The numbers along the base of the sequence logo indicate the 5′, middle, and 3′ nucleotides of the triplet DNA binding site for finger 2. The height of each nucleotide at each position of the triplet DNA binding site is determined by multiplying the relative DNA binding affinity of the nucleotide by the total information at that position, so that a taller printed nucleotide is more beneficial for tight binding than a shorter one. The nucleotides are sorted so that the nucleotide that is most beneficial for tight binding is on top. The values along the y-axis indicate the number of bits of information at each position of the triplet DNA binding site. (A) wt. (B) RGPD. (C) REDV. (D) LRHN. (E) KASN.

FIG. 3. Evolution of sequence-specific DNA binding zinc fingers from selections of the phage display library. Phage pools isolated from different rounds of selections analyzed using DNA microarrays. One of nine replicates on each microarray slide is shown for each of the binding experiments described. Spots with high relative signal intensities in each of the rounds are labeled to indicate the bound DNA sequence. (A) Rounds 2-4 of the selection using the middle triplet GCG. Round 1 (not shown) did not have any outstanding spots. Round 2 shows binding to the wildtype Zif268 DNA binding site, which is spotted at a high concentration on the periphery of the array (62). Phage binding to this DNA sequence are lost in subsequent rounds of selection. (B) Rounds 1-3 of the selection using the middle triplet TCC. Round 1 did not have any outstanding spots. (C) Portions of the sequences present at the GAC and TCC spots on the microarrays. The nine bp binding sites for variant zinc finger phage are underlined, and the triplet binding sites for finger 2 are boldfaced. Note that the sequence of the bottom strand of the TCC oligonucleotide closely resembles the sequence of the top strand of the GAC oligonucleotide.

FIG. 4. Relationship between relative fluorescence intensity and DNA binding affinity. (A) SybrGreenI-stained microarray. (B) Low laser power scan of wildtype Zif268 bound to a microarray. (C) High laser power scan of wildtype Zif268 bound to a microarray. White pixels indicate saturated signal intensity. (D) Plot showing the relationship between relative signal intensity and K_{d}^{app}. Error bars indicate 1 standard deviation of the SybrGreen I normalized binding data.

FIG. 5. Binding of the entire zinc finger phage display library to a microarray indicates that DNA triplets with a 5′ T or G are bound preferentially over triplets with a 5′ A or C. The data are plotted in such a way as to analyze binding as a function of the 5′ nucleotide. The average relative fluorescence intensity of all 64 different triplet binding sites was normalized to 1; therefore, a value less than 1 indicates that the particular sequence is bound less than average, and a value greater than 1 indicates that the particular sequence is bound greater than average.

### DETAILED DESCRIPTION

The present invention utilizes DNA microarray technologies for a highly parallel method of studying the sequence specificity of DNA-protein interactions (M. L. Bulyk, E. Gentalen, D. J. Lockhart, G. M. Church, Nature Biotechnol. 17, 573 (1999); Shena, supra.).

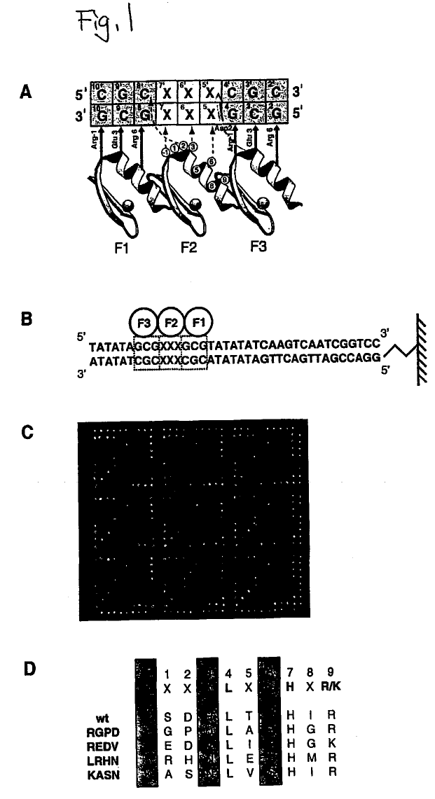

Types of DNA binding proteins. These experiments are not limited to zinc finger proteins, as other structural classes of DNA binding domains have been displayed on the surface of phage, including homeodomains, helix-tum-helix motifs, beta-sheets, leucine zippers, and steroid receptors (J. Connolly, J. Augustine, C. Francklyn, Nucleic Acids Res. 27, 1182 (1999); E. d'Alencon, S. Ehrlich, J. Bacteriol. 182, 2973 (2000); B. Ruan, J. Hoskins, L. Wang, P. Bryan, Protein Sci. 7,2345 (1998); R. Crameri, M. Suter, Gene 137, 69 (1993); S. Chusacultanachai, et al., J. Biol. Chem. 274, 23591 (1999)). DNA microarrays are used to study DNA recognition by the zinc finger, since this domain is one of the most common structural motifs found in eukaryotic transcription factors (C. O. Pabo, R. T. Sauer, Annu. Rev. Biochem. 61, 1053 (1992)). An important member of the Cys₂His₂ class of this family of proteins is the mouse transcription factor Zif268. Zif268 serves as a valuable model system for studying zinc finger-DNA recognition, since crystallographic data of the Zif268 DNA-protein complex is available (N. P. Pavletich, C. O. Pabo, Science 252, 809 (1991); M. Elrod-Erickson, M. Rould, L Nekludova, C. Pabo, Structure 4, 1171 (1996)). A simple model of the DNA-protein interactions has the three fingers in the DNA binding domain binding as independent modules to three tandem 3 bp subsites (FIG. 1A). This modularity has been exploited in studies aimed at unraveling the rules governing the interactions between zinc finger residues and the DNA bases they contact (M. Suzuli, N. Yagi, Proc. Natl. Acad. Sci. U.S.A. 91, 12357 (1994); Y. Choo, A. Klug, Curr. Opin. Struct. Biol. 7, 117 (1997)).

Furthermore, epitope-tagged DNA binding proteins or whole transcription factors could be used instead of displaying just the proteins on the surface of phage.

All patents and references cited herein are incorporated in their entirety by reference.

Double-Stranded Bimolecular Nucleic Acid Arrays

Methods and applications for double-stranded nucleic acid arrays are described in PCT WO99/19510, the disclosure of which is incorporated herein by reference

I. Preparation of an Array of Immobilized First Nucleic Acid Strands

Synthesis of a nucleic acid array useful according to the present invention is a bipartite process, which entails the production of a diverse array of single-stranded nucleic acid molecules that are immobilized on the surface of a solid support matrix, followed by priming and enzymatic synthesis of a second nucleic acid strand, comprising either RNA or DNA. A highly preferred method of carrying out synthesis of the immobilized single-stranded array is that of Lockhart, described in U.S. Pat. No. 5,556,752 the contents of which are herein incorporated by reference. Of the methods described therein, that which is of particular use describes the synthesis of such an array on the surface of a single solid support having a plurality of preselected regions. A method whereby each chemically-distinct feature of the array is synthesized on a separate solid support is also described by Lockhart. These methods, and others, are briefly summarized below.

The solid support may comprise biological, nonbiological, organic or inorganic materials, or a combination of any of these. It is contemplated that such materials may exist as particles, strands, precipitates, gels, sheets, tubing, spheres, containers, capillaries, pads, slices, films, plates or slides. Preferably the solid support takes the form of plates or slides, small beads, pellets, disks or other convenient forms. It is highly preferred that at least one surface of the support is substantially flat. The solid support may take on alternative surface configurations. For example, the solid support may contain raised or depressed regions on which synthesis takes place. In some instances, the solid support will be chosen to provide appropriate light-absorbing characteristics. For example, the support may be a polymerized Langmuir Blodgett film, functionalized glass, Si, Ge, GaAs, GaP, SiO₂, SiN₄, modified silicon, or one of a variety of gels or polymers such as (poly)tetrafluoroethylene, (poly)vinylidendifluoride, polystyrene, polycarbonate, or combinations thereof. Other suitable solid support materials may be used, and will be readily apparent to those of skill in the art. Preferably, the surface of the solid support will contain reactive groups, which could be carboxyl, amino, hydroxyl, thiol, or the like. More preferably, the surface will be optically transparent and will have surface Si—OH functionalities, such as are found on silica surfaces.

According to the invention, a first nucleic acid strand is anchored to the solid support by as little as an intermolecular covalent bond. Alternatively, a more elaborate linking molecule may attach the nucleic acid strand to the support. Such a molecular tether may comprise a surface-attaching portion which is directly attached to the solid support. This portion can be bound to the solid support via carbon-carbon bonds using, for example, supports having (poly)trifluorochloroethylene surfaces, or preferably, by siloxane bonds (using, for example, glass or silicon oxide as the solid support). Siloxane bonds with the surface of the support can be formed via reactions of surface attaching portions bearing trichlorosilyl or trialkoxysilyl groups. The surface attaching groups will also have a site for attachment of the longer chain portion. It is contemplated that suitable attachment groups may include amines, hydroxyl, thiol, and carboxyl groups. Preferred surface attaching portions include aminoalkylsilanes and hydroxyalkylsilanes. It is particularly preferred that the surface attaching portion of the spacer is selected from the group comprising bis(2-hydroxyethyl)-aminopropyltriethoxysilane, 2-hydroxyethylaminopropyltriethoxysilane, aminopropyltriethoxysilane and hydroxypropyltriethoxysilane.

The longer chain portion of the spacer can be one of a variety of molecules which are inert to the subsequent conditions for polymer synthesis, examples of which include: aryl acetylene, ethylene glycol oligomers containing 2-14 monomer units, diamines, diacids, amino acids, peptides, or combinations thereof. It is contemplated that the longer chain portion is a polynucleotide. The longer chain portion which is to be used as part of the spacer can be selected based upon its hydrophilic/hydrophobic properties to improve presentation of the double-stranded oligonucleotides to certain receptors, proteins or drugs. It can be constructed of polyethyleneglycols, polynucleotides, alkylene, polyalcohol, polyester, polyamine, polyphosphodiester and combinations thereof.

Additionally, for use in synthesis of the arrays of the invention, the spacer will typically have a protecting group, attached to a functional group (i.e., hydroxyl, amino or carboxylic acid) on the distal or terminal end of the chain portion (opposite the solid support). After deprotection and coupling, the distal end is covalently bound to an oligomer. As used in discussion of the spacer region, the term “alkyl” refers to a saturated hydrocarbon radical which may be straight -chain or branced-chain (for example, ethyl,isopropyl, t-amyl, or 2,5-0dimethylhexyl). When “alkyl” or “alkylene” is used to refer to a linking group or a spacer, it is taken to be a group having two available valences for covalent attachment, for example, —CH₂CH₂—, —CH₂CH₂CH₂—, —CH₂CH₂CH(CH₃)CH₂— —CH₂(CH₂CH₂)₂CH₂—. Preferred alkyl groups as substitutents are those containing 1 to 10 carbon atoms, with those containing 1 ato 6 carbon atoms being particularly preferred. Preferred alkyl or alkylene groups as linking groups are those containing 1 to 20 carbon atoms, with those containing 3 to 6 carbon atoms being particularly preferred. The term “polyethylene glycol” is used to refer to those molecules which have repeating units of ethylene glycol, for example, hexaethylene glycol (HO—(CH₂CH₂O)₅—CH₂(CH₂CH₂OH). When the term “polyethylene glycol” is used to refer to linking groups and spacer groups, it would be understood by one of skill in the art that other polyethers of polyols could be used as well (i.e., polypropylene glycol or mistures of ethylene and propeylene glycols).

The term “protecting group”, as used herein, refers to any of the groups which are designed to block one reactive site in a molecule while a chemical reaction is carried out at another reactive site. More particularly, the protecting groups used herein can be any of those groups described in Greene et al., 1991, Protective Groups In Organic Chemistry, 2nd Ed., John Wiley & Sons, New York, N.Y, incorporated herein by reference. The proper selection of protecting groups for a particular synthesis will be governed by the overall methods employed in the synthesis. For example, in “light-directed” synthesis, discussed below, the protecting groups will be photolabile protecting groups, e.g. NVOC and MeNPOC. In other methods, protecting groups may be removed by chemical methods and include groups such as FMOC, DMT and others known to those of skill in the art.

a. Nucleic Acid Arrays on a Single Support

1. Light-Directed Methods

Where a single solid support is employed, the oligonucleotides of the present invention can be formed using a variety of techniques known to those skilled in the art of polymer synthesis on solid supports. For example, “light-directed” methods are described in U.S. Pat. No. 5,143,854 and U.S. Pat. No. 5,510,270 and U.S. Pat. No. 5,527,681. These methods, involve activating predefined regions of a solid support and then contacting the support with a preselected monomer solution. These regions can be activated with a light source, typically shown through a mask (much in the manner of photolithography techniques used in integrated circuit fabrication). Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. Other steps, such as washing unreacted monomer solution from the support, can be used as necessary. Other applicable methods include mechanical techniques such as those described in PCT No.92/10183, U.S. Pat. No. 5,384,261 also incorporated herein by reference for all purposes. Still further techniques include bead based techniques such as those described in PCT US/93/04145, also incorporated herein by reference, and pin based methods such as those described in U.S. Pat. No. 5,288,514, also incorporated herein by reference.

The surface of a solid support, optionally modified with spacers having photolabile protecting groups such as NVOC and MeNPOC, is illuminated through a photolithographic mask, yielding reactive groups (typically hydroxyl groups) in the illuminated regions. A 3′-O-phosphoramidite activated deoxynucleoside (protected at the 5′-hydroxyl with a photolabile protecting group) is then presented to the surface and chemical coupling occurs at sites that were exposed to light. Following capping and oxidation, the support is rinsed and the surface illuminated through a second mask, to expose additional hydroxyl groups for coupling. A second 5′-protected, 3′-O-phosphoramidite activated deoxynucleoside is presented to the surface. The selective photodeprotection and coupling cycles are repeated until the desired set of oligonucleotides is produced. Alternatively, an oligomer of from, for example, 4 to 30 nucleotides can be added to each of the preselected regions rather than synthesize each member one nucleotide monomer at a time.

2. Flow Channel or Spotting Methods

Additional methods applicable to array synthesis on a single support are described in U.S. Pat. No. 5,384,261, incorporated herein by reference for all purposes. In the methods disclosed in these applications, reagents are delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites.

A typical “flow channel” method applied to arrays of the present invention can generally be described as follows: Diverse polymer sequences are synthesized at selected regions of a solid support by forming flow channels on a surface of the support through which appropriate reagents flow or in which appropriate reagents are placed. For example, assume a monomer “A” is to be bound to the support in a first group of selected regions. If necessary, all or part of the surface of the support in all or a part of the selected regions is activated for binding by, for example, flowing appropriate reagents through all or some of the channels, or by washing the entire support with appropriate reagents. After placement of a channel block on the surface of the support, a reagent having the monomer A flows through or is placed in all or some of the channel(s). The channels provide fluid contact to the first selected regions, thereby binding the monomer A to the support directly or indirectly (via a spacer) in the first selected regions.

Thereafter, a monomer B is coupled to second selected regions, some of which may be included among the first selected regions. The second selected regions will be in fluid contact with a second flow channel(s) through translation, rotation, or replacement of the channel block on the surface of the support; through opening or closing a selected valve; or through deposition of a layer of chemical or photoresist. If necessary, a step is performed for activating at least the second regions. Thereafter, the monomer B is flowed through or placed in the second flow channel(s), binding monomer B at the second selected locations. In this particular example, the resulting sequences bound to the support at this stage of processing will be, for example, A, B, and AB. The process is repeated to form a vast array of sequences of desired length at known locations on the support.

After the support is activated, monomer A can be flowed through some of the channels, monomer B can be flowed through other channels, a monomer C can be flowed through still other channels, etc. In this manner, many or all of the reaction regions are reacted with a monomer before the channel block must be moved or the support must be washed and/or reactivated. By making use of many or all of the available reaction regions simultaneously, the number of washing and activation steps can be minimized. One of skill in the art will recognize that there are alternative methods of forming channels or otherwise protecting a portion of the surface of the support. For example, a protective coating such as a hydrophilic or hydrophobic coating (depending upon the nature of the solvent) is utilized over portions of the support to be protected, sometimes in combination with materials that facilitate wetting by the reactant solution in other regions. In this manner, the flowing solutions are further prevented from passing outside of their designated flow paths.

The “spotting” methods of preparing compounds and arrays of the present invention can be implemented in much the same manner. A first monomer, A, can be delivered to and coupled with a first group of reaction regions which have been appropriately activated. Thereafter, a second monomer, B, can be delivered to and reacted with a second group of activated reaction regions. Unlike the flow channel embodiments described above, reactants are delivered in relatively small quantities by directly depositing them in selected regions. In some steps, the entire support surface can be sprayed or otherwise coated with a solution, if it is more efficient to do so. Precisely measured aliquots of monomer solutions may be deposited dropwise by a dispenser that moves from region to region. Typical dispensers include a micropipette to deliver the monomer solution to the support and a robotic system to control the position of the micropipette with respect to the support, or an ink-jet printer. In other embodiments, the dispenser includes a series of tubes, a manifold, an array of pipettes, or the like so that various reagents can be delivered to the reaction regions simultaneously.

3. Pin-Based Methods

Another method which is useful for the preparation of the immobilized arrays of single-stranded DNA molecules X of the present invention involves “pin-based synthesis.” This method, which is described in detail in U.S. Pat. No. 5,288,514, previously incorporated herein by reference, utilizes a support having a plurality of pins or other extensions. The pins are each inserted simultaneously into individual reagent containers in a tray. An array of 96 pins is commonly utilized with a 96-container tray, such as a 96-well microtitre dish. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously. The invention provides for the use of support(s) on which the chemical coupling steps are conducted. The support is optionally provided with a spacer, S, having active sites. In the particular case of oligonucleotides, for example, the spacer may be selected from a wide variety of molecules which can be used in organic environments associated with synthesis as well as aqueous environments associated with binding studies such as may be conducted between the nucleic acid members of the array and other molecules. These molecules include, but are not limited to, proteins (or fragments thereof), lipids, carbohydrates, proteoglycans and nucleic acid molecules. Examples of suitable spacers are polyethyleneglycols, dicarboxylic acids, polyamines and alkylenes, substituted with, for example, methoxy and ethoxy groups. Additionally, the spacers will have an active site on the distal end. The active sites are optionally protected initially by protecting groups. Among a wide variety of protecting groups which are useful are FMOC, BOC, t-butyl esters, t-butyl ethers, and the like.

Various exemplary protecting groups are described in, for example, Atherton et al., 1989, Solid Phase Peptide Synthesis, IRL Press, incorporated herein by reference. In some embodiments, the spacer may provide for a cleavable function by way of, for example, exposure to acid or base.

b. Arrays on Multiple Supports

Yet another method which is useful for synthesis of compounds and arrays of the present invention involves “bead based synthesis.” A general approach for bead based synthesis is described in PCT/US93/04145 (filed Apr. 28, 1993), the disclosure of which is incorporated herein by reference.

For the synthesis of molecules such as oligonucleotides on beads, a large plurality of beads are suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and redistributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. As pointed out by Lockhart (U.S. Pat. No. 5,556,752) an individual bead may be tagged with a sequence which is unique to the double-stranded oligonucleotide thereon, to allow for identification during use.

II. Preparation of Oligonucleotide Primers

Oligonucleotide primers useful to synthesize bimolecular arrays are single-stranded DNA or RNA molecules that are hybridizable to a nucleic acid template to prime enzymatic synthesis of a second nucleic acid strand. The primer may therefore be of any sequence composition or length, provided it is complementary to a portion of the first strand.

It is contemplated that such a molecule is prepared by synthetic methods, either chemical or enzymatic. Alternatively, such a molecule or a fragment thereof may be naturally occurring, and may be isolated from its natural source or purchased from a commercial supplier. It is contemplated that oligonucleotide primers employed in the present invention will be 6 to 100 nucleotides in length, preferably from 10 to 30 nucleotides, although oligonucleotides of different length may be appropriate.

Additional considerations with respect to design of a selected primer relate to duplex formation, and are described in detail in the following section.

III. Primed Enzymatic Second-Strand Nucleic Acid Synthesis to Form a Double-Stranded Array

Of central importance in carrying out preparation of a bimolecular array is selective hybridization of an oligonucleotide primer to the first nucleic acid strand in order to permit enzymatic synthesis of the second nucleic acid strand. Any of a number of enzymes well known in the art can be utilized in the synthesis reaction. Preferably, enzymatic synthesis of the second strand is performed using an enzyme selected from the group comprising DNA polymerase I (exo⁽⁻⁾ Klenow fragment), T4 DNA polymerase, T7 DNA polymerase, modified T7 DNA polymerase, Taq DNA polymerase, exo⁽⁻⁾ vent DNA polymerase, exo⁽⁻⁾ deep vent DNA polymerase, reverse transcriptase and RNA polymerase.

Typically, selective hybridization will occur when two nucleic acid sequences are substantially complementary (typically, at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference. As a result, it is expected that a certain degree of mismatch at the priming site can be tolerated. Such mismatch may be small, such as a mono-, di- or tri-nucleotide. Alternatively, it may encompass loops, which we define as regions in which mismatch encompasses an uninterrupted series of four or more nucleotides. Note that such loops within the oligonucleotide priming site are encompassed by the present invention; however, the invention does not provide double-stranded nucleic acids that comprise loop structures between the 5′ end of the first strand and the 3′ end of the second strand. In addition, loop structures outside the priming site, but which do not encumber the 5′ end of the first strand or the 3′ end of the second strand are not provided by the present invention, since there is no known mechanism for generating such structures in the course of enzymatic second-strand nucleic acid synthesis. Both the 5′ end of the first strand and the 3′ end of the second strand must be free of attachment to each other via a continuous single strand.

Either strand may comprise RNA or DNA. Overall, five factors influence the efficiency and selectivity of hybridization of the primer to the immobilized first strand. These factors are (i) primer length, (ii) the nucleotide sequence and/or composition, (iii) hybridization temperature, (iv) buffer chemistry and (v) the potential for steric hindrance in the region to which the probe is required to hybridize.

There is a positive correlation between primer length and both the efficiency and accuracy with which a primer will anneal to a target sequence; longer sequences have a higher T_{M} than do shorter ones, and are less likely to be repeated within a given first nucleic acid strand, thereby cutting down on promiscuous hybridization. Primer sequences with a high G-C content or that comprise palindromic sequences tend to self-hybridize, as do their intended target sites, since unimolecular, rather than bimolecular, hybridization kinetics are genererally favored in solution; at the same time, it is important to design a primer containing sufficient numbers of G-C nucleotide pairings to bind the target sequence tightly, since each such pair is bound by three hydrogen bonds, rather than the two that are found when A and T bases pair. Hybridization temperature varies inversely with primer annealing efficiency, as does the concentration of organic solvents, e.g. formamide, that might be included in a hybridization mixture, while increases in salt concentration facilitate binding. Under stringent hybridization conditions, longer probes must be used, while shorter ones will suffice under more permissive conditions. Stringent hybridization conditions will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As several factors may affect the stringency of hybridization, the combination of parameters is more important than the absolute measure of any one alone.

Primers must be designed with the above first four considerations in mind. While estimates of the relative merits of numerous sequences can be made mentally, computer programs have been designed to assist in the evaluation of these several parameters and the optimization of primer sequences. Examples of such programs are “PrimerSelect” of the DNAStar™ software package (DNAStar, Inc.; Madison, Wis.) and OLIGO 4.0 (National Biosciences, Inc.). Once designed, suitable oligonucleotides may be prepared by the phosphoramidite method described by Beaucage and Carruthers, 1981, Tetrahedron Lett., 22: 1859-1862, or by the triester method according to Matteucci et al., 1981, J. Am. Chem. Soc., 103: 3185, both incorporated herein by reference, or by other chemical methods using either a commercial automated oligonucleotide synthesizer or light directed methods as mentioned herein.

Generating A Microarray

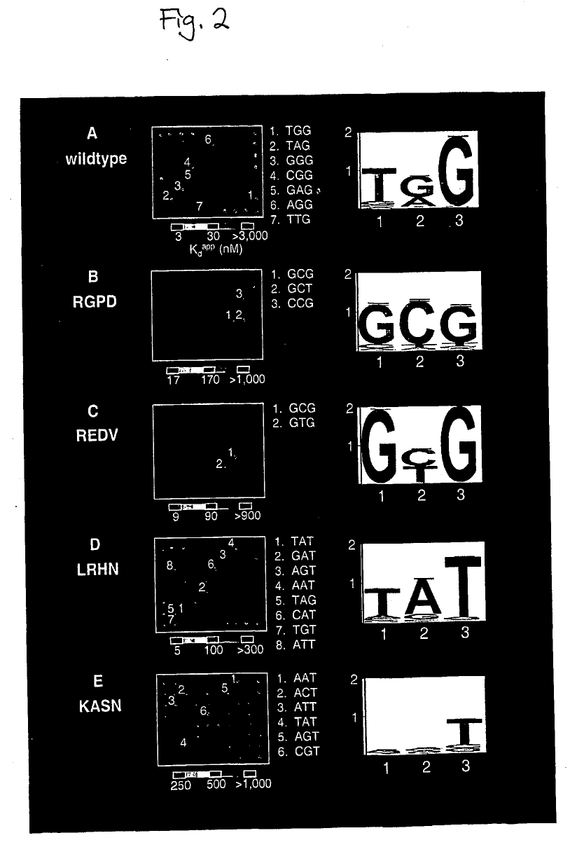

DNA microarrays were used to examine the spectrum of binding site specificitie of a collection of Zif268 mutants selected from a phage display library of the second finger. Quantitative measurements of more than 750 DNA-protein interactions were gathered from 10 different microarray binding assays using wildtype Zif268, four mutants, and seven pools of mutants (FIGS. 2A-E, 3A,B). Double-stranded DNAs containing the wildtype binding sites for fingers 1 and 3 and all possible triplet binding sites for finger 2 of wildtype Zif268 were created by primer extension on unique oligonucleotides using a universal primer (FIG. 1B).

The following Cy3-labeled oligonucleotide (Operon) was spotted at 10 μM in 150 mM K₂HPO₄, pH 9.0 for alignment purposes: 5′ TCAGAACTCACCTGTTAGAC 3′. The following set of 64 oligonucleotides 37 nt in length was synthesized (Operon) so as to represent all possible 3 nt central finger sites for Zif268 zinc fingers: 5′ TATATAGCGNNNGCGTATATATCAAGTCAATCGGTCC 3′ (the 3 sites for fingers 1 through 3 are underlined; bold letters show the position of the 64 possible 3 nt sites for the central finger). The following 16mer was synthesized with a 5′ amino linker (Operon) and used as a universal primer: 5′ GGACCGATTGACITGA 3′. Each of the 64 unmodified 37mers was combined with the amino-tagged 16mer in a 2:1 molar ratio in a Sequenase reaction using 20 μM 16mer. The completed extension reactions were exchanged into 150 mM K₂BPO₄, pH 9.0 using CentriSpin-10 spin columns (Princeton Separations, Inc.). The resulting samples were transferred to a 384-well plate for arraying.

Nine replicates of each of these 64 different sequences were printed onto glass slides. Glass slides (Gold Seal) were cleaned for 0.5 to 2 hrs in 2 N nitric acid. After rinsing in distilled water, the slides were soaked in distilled water for 5 to 15 minutes, and then washed once with acetone. The slides were silanized by immersing them for 15 minutes in a solution of 1% aminopropyl-methyl-diethoxysilane (Fluka) dissolved in 95% acetone. After washing the slides twice in acetone, the slides were baked for 30 minutes at 75° C. The surface of the slides was then activated by placing the slides in a solution of 0.5% 1,4-diphenylene-diisothiocyanate (PDC) (Fluka) dissolved in a solution consisting of 40 ml pyridine and 360 ml anhydrous N,N-dimethylformamide for 2 to 4 hrs. The slides were then washed twice with methanol, twice with acetone, and stored in a dessicator until use. A custom-built arraying robot equipped with piezo-electric printheads was used to print the microarrays. After printing, the microarrays were incubated overnight at room temperature, then for 1 hr at 37° C. in a humidity chamber containing 300 mM K₂HPO₄, pH 9.0. The rest of the PDC surface was inactivated by a 10 min incubation in 1% ammonium hydroxide/0.1% SDS/200 mM NaCl. After washing in 4×SSC, the slides were neutralized in 6×SSPE/0.01% Triton X-100, washed twice in 4×SSC, then washed in 2×SSC and spun dry in a clinical centrifuge. Slides were stored in a closed box at room temperature until use.

Verifying Chip Quality

To normalize for possible variability in the DNA concentrations of the different DNA samples that were spotted onto the microarrays, separate microarrays manufactured in the same print run were quantified by SybrGreen I staining.

Microarrays were stained by incubation in a 1:5000 dilution of SybrGreen I (Molecular Probes) in 2×SSC/0.1% Triton, then washed in 2×SSC/0.1% Triton, followed by a wash in 2×SSC.

For a microarray stained with SybrGreen I, the average signal intensities of all 64 different triplet sequences were normalized to 1. The standard deviation of these 64 different sequences was 0.067. The average standard deviation of any particular sequence over all nine replicates on the microarrays was 0.108.

Detecting Zinc Finger Binding to a Microarray

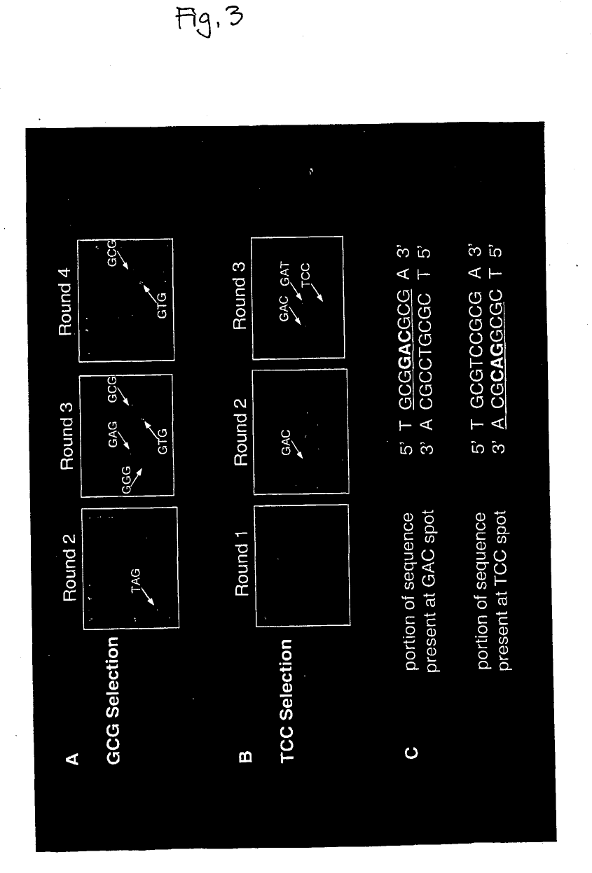

DNA microarrays were used examine the spectrum of binding site specificities of a collection of Zif268 mutants selected from a phage display library of the second finger. Quantitative measurements of more than 750 DNA-protein interactions were gathered from 10 different microarray binding assays using wildtype Zif268, four mutants, and seven pools of mutants (see below, FIGS. 2A-E, FIGS. 3A,B). Phage displaying the three Zif268 zinc fingers were harvested directly from bacterial cultures and bound to the microarrays. The bound zinc finger phage were labeled fluorescently using a primary antibody against a phage coat protein and an R-phycoerythrin-conjugated secondary antibody (FIG. 1C).

For production of Zif phage, overnight bacterial cultures of TG1 (or JM109) cells, each producing a particular zinc finger phage or pool of phages, were grown at 30° C. in 2×TY medium containing 50 μM zinc acetate and 15 μg/ml tetracycline (2×TY/Zn/Tet). Culture supernatants containing phage were diluted 2-fold by addition of PBS/Zn containing 4% (w/v) nonfat dried milk, 2% (v/v) Tween 20 and 100 μg/ml salmon testes DNA (Sigma). The slides were blocked with 2% milk in PBS/Zn for 1 hr, then washed once with PBS/Zn/0.1% Tween 20, then once with PBS/Zn/0.01% Triton X-100. The diluted phage solutions were then added to the slides and binding was allowed to proceed for 1 hr. The slides were then washed 5 times with PBS/Zn/1% Tween 20, and then 3 times with PBS/Zn/0.01% Triton X-100. Rabbit anti(M13) antibody (Pharmacia) was diluted in PBS/Zn containing 2% milk, pre-incubated for at least 1 hr, and added to the slide. After incubation for 1 hr at room temperature, the slide was washed 3 times with PBS/Zn/0.05% Tween 20, and 3 times with PBS/Zn/0.01% Triton X-100. R-phycoerythrin-conjugated goat anti(rabbit IgG) (Sigma) was diluted in PBS/Zn containing 2% milk, pre-incubated for at least 1 hr, and added to the slide. After incubation for 1 hr at room temperature, the slide was washed 3 times with PBS/Zn/0.05% Tween 20, 3 times with PBS/Zn/0.01% Triton X-100, and once with PBS/Zn, and then scanned.

Each of the DNA concentration-normalized fluorescence intensities was expressed as a fraction of the fluorescence intensity of the DNA sequence with the highest average intensity for the particular Zif268 mutant being examined.

Microarrays were scanned using a GSI Lumonics ScanArray 5000 microarray scanner. Images were scanned at a resolution of 10 μm per pixel. Fluorescent signals were detected using a helium neon laser with an excitation of 543.5 nm and a 570 nm bandpass filter for R-phycoerythrin and Cy3, and an argon laser with an excitation of 488 nm and a 522 nm bandpass filter for SybrGreen I. The signal intensities of each of the spots in the scanned images were quantified using ImaGene version 3.0 software (BioDiscovery). Subsequent analyses were performed with Perl scripts. Writing Perl scripts is routine for a skilled artisan (Wall et al. (2000) Programming Perl, 3rd Edition. O'Reilly & Associates, Inc.; http://bio.perl.org/).

Background signal intensities were calculated individually for each spot as the area of the spot multiplied by the median signal intensity in a 5 pixel thick perimeter at a distance of 5 pixels outside of each spot. After background subtraction, the relative signal intensity of each of the spots within a replicate was calculated as a fraction of the highest signal intensity for a spot containing one of the 64 different 37 bp sequences. The relative intensities were calculated individually within each replicate before averaging over all the replicates on the microarray so as to control for any overall variation in the binding and antibody reactions. Each of these relative signal intensities was then averaged over the 9 replicates present on each slide. To normalize for the amount of DNA present in each spot, each of the average relative signal intensities from zinc finger phage binding were divided by each of the respective average relative signal intensities from SybrGreen I staining.

Ouantitating Zinc Finger Binding

As a validation of this protocol, wildtype Zif268 phage were bound to a microarray (FIGS. 1C, 2A). To ensure that all the binding affinity data were calculated using fluorescence intensities below the saturation level of the microarray scanner, the microarrays were scanned at multiple laser power settings. The relative fluorescence intensities for each scan were then normalized relative to a sequence with one of the highest fluorescence intensities on the respective scans. These ratios were then multiplied to calculate all the fluorescence intensities as a fraction of the sequence with the overall highest fluorescence intensity. The dynamic range of relative fluorescence intensities spanned 2 orders of magnitude and corresponded to a dynamic range of apparent binding constants (K_{d}^{app}'s) that spanned at least 3 orders of magnitude. For the microarray binding experiment using wildtype Zif268, the highest relative signal intensity observed was 1 for the triplet TGG, and the lowest relative fluorescence intensity observed was 0.0305 for the triplet AGA.

To determine apparent dissociation constants (K_{d}^{app}'s) ELISAs were performed at a series of DNA concentrations, and then the K_{d}^{app}'s were estimated from a plot of A₄₅₀ versus DNA concentration. These ELISAs were performed in at least triplicate. Unmodified DNA oligonucleotides corresponding to the sequences on the chips were synthesized (Operon) and then double-stranded using biotin-tagged universal 16mers in Sequenase reactions. For production of Zif phage, overnight bacterial cultures of TG1 (or JM109) cells, each containing a particular zinc finger phage or pool of phage, were grown at 30° C. in 2×TY/Zn/Tet. DNA solutions were diluted to a final concentration of 4% (w/v) nonfat dried milk and 2% (v/v) Tween 20 using 8% (w/v) nonfat dried milk and 4% (v/v) Tween 20. Serial two-fold dilutions were made with PBS/Zn containing 4% (w/v) nonfat dried milk and 2% (v/v) Tween 20. These dilutions were then diluted a further two-fold with an equal volume of culture supernatant containing phage. All binding reactions contained 50 μg/ml salmon testes DNA, as in the microarray binding experiments. Binding reactions were equilibrated at room temperature for 1 hr, then bound to high capacity streptavidin-coated microtiter plates for 1 hr (Boehringer Mannheim). Binding reactions were removed from the wells, and the wells were washed 5 times with PBS/Z-n1% Tween 20, and then 3 times with PBS/Zn/0.01% Triton X-100. Because the binding reactions were washed, the K_{d}^{app}'s represent non-equilibrium binding constants. Horseradish peroxidase (HRP) conjugated anti-(M13)-antibody (Pharmacia) was diluted 1:5000 in PBS/Zn containing 2% milk and pre-incubated for at least 15 min. The HRP-antibody dilution was then added to the wells and allowed to incubate for 1 hr. The antibody solution was removed from the wells, and then the wells were washed 3 times with PBS/Zn/0.05% Tween 20,3 times with PBS/Zn/0.01% Triton X-100, then developed using 3,3′,5,5-tetramethylbenzidine (B) (Sigma) and 1M H₂SO₄. The colorimetric signal was quantified at A₄₅₀ with Softmax on a Vmax Kinetic Microplate Reader (Molecular Devices) and plotted as a function of DNA concentration using Microsoft Excel. All K_{d}^{app}'s were determined using binding reactions that were well within the biotin binding capacity of the streptavidin-coated microtiter plates. The DNA concentration which achieved half-maximal A₄₅₀ was used to calculate the K_{d}^{app}. Since these measurements provide apparent, not actual K_{d}'s, all final observed K_{d}^{app} values were scaled by the same constant so that the K_{d}^{app} for wildtype Zif268 with the triplet TGG was equal to 3.0 nM.

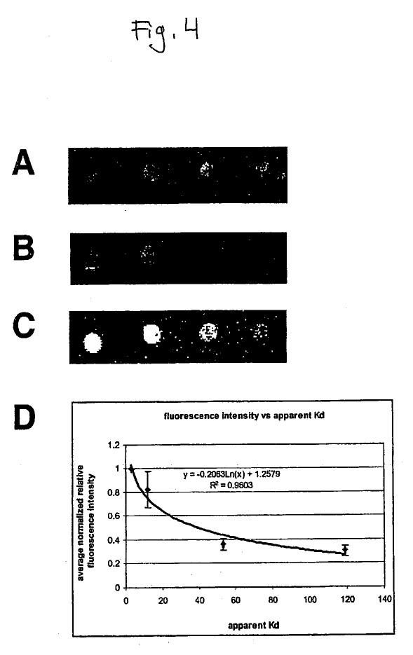

To evaluate the relationship between the normalized fluorescence intensities and the DNA binding affinities of the zinc fingers, the binding affinities of wildtype Zif268 phage for a set of DNA sequences were determined by performing zinc finger phage ELISA at a series of DNA concentrations (Griffiths et al., supra.). These sequences were chosen because they span a range of relative fluorescence intensities. For this evaluation a separate set of microarrays were spotted with these DNA sequences. Since some of these sequences contain mutations in the triplet binding sites for fingers 1 and 3 of Zif268, they were not printed on the microarrays containing all different triplet binding sites for finger 2. The relative fluorescence intensities were found to correlate well with the natural logarithm of the K_{d}^{app}'s (FIG. 4) Linear, exponential, and logarithmic functions were fitted to the data, and it was found that the data best fit a natural logarithm function. The R-squared values of the natural logarithm plots for the Zif phage variants are 99.7 for wildtype Zif268, 99.7 for RGPD, 96.6 for LRHN, 92.5 for KASN, and 81.4 for REDV. Since REDV had only 2 high affinity triplet binding sites, it was not possible to construct a plot with more points on it to achieve a higher R-squared value.

Therefore for each variant Zif phage a calibration curve was constructed by determining the K_{d}^{app}'s of a few representative sequences that spanned the range of relative fluorescence intensities on the microarrays spotted with all different triplet binding sites for finger 2. These calibration curves were used to interpolate the K_{d}^{app}'s for the remaining sequences on the microarrays (Table 1A). These binding site preferences were then weighted according to their K_{d}^{app}'s, and used to construct sequence logo representations of each variant's binding site profile (T. D. Schneider, G. D. Stormo, L. Gold, J. Mol. Biol. 188, 415 (1986). For each variant, either the top 15 sequences (RGPD, LRHN, KASN), or all sequences with an K_{d}^{app} at least 1% as strong as the sequence with the highest binding affinity (wildtype Zif268, REDV), were used to generate the sequence logos (FIGS. 2A-E). Each of these input sequences was weighted according to the inverse of its K_{d}^{app}, so that the sequences with the highest binding affinities had the greatest contribution in creation of the sequence logo.

ZIF268 Binding Specificity—Ex I: Specific Binding

TABLE 1A

| sequence of finger 2 | relative | |
|---|---|---|
| DNA binding site | signal intensity | K_{d}^{app} (nM) |
| TGG (wildtype) | 1 | 3.0 +/− 0.57 |
| TAG | 0.751 +/− 0.214 | 6.7 +/− 1.6 |
| GGG | 0.372 +/− 0.111 | 28 |
| CGG | 0.320 +/− 0.075 | 35 |
| AGG | 0.256 +/− 0.105 | 54 +/− 3.3 |
| TTG | 0.153 +/− 0.037 | 71 +/− 9.8 |
| GAG | 0.150 +/− 0.029 | 75 |
| TCG | 0.085 +/− 0.025 | 180 |
| CAG | 0.068 +/− 0.018 | 380 +/− 67 |
| AAA (neg. control) | 0.052 +/− 0.015 | >5000 |

Apparent K_{d} values of the binding of zinc finger phage to DNA containing variants of the Zif268 binding sites with mutated middle triplets. (A) wildtype Zif268. (B) RGPD and REDV. (C) LRHN. (D) KASN. Boldfaced K_{d}^{app} values were determined by phage ELISA; italicized values were interpolated from a plot of relative signal intensity versus K_{d}^{app}. The mean and standard deviation values are listed in the K_{d}^{app} column.

Distinguishing Binding Site Preferences—Ex II: Specific Binding

To determine if proteins with overlapping binding site preferences could be distinguished, two related Zif268 mutants were bound to microarrays (FIGS. 2B,C; Table 1B).

TABLE 1B

| sequence of | RGPD | RGPD | REDV | REDV |
|---|---|---|---|---|
| finger 2 | relative signal | K_{d}^{app} | relative signal | K_{d}^{app} |
| DNA binding site | intensity | (nM) | intensity | (nM) |
| GCG | 1 | 17 +/− 4.0 | 1 | 11 +/− 4.3 |
| GCT | 0.406 +/− 0.117 | 220 | 0.077 +/− 0.005 | 1600 |
| CCG | 0.328 +/− 0.094 | 250 +/− 25 | 0.171 +/− 0.038 | 880 +/− 98 |
| GCA | 0.216 +/− 0.017 | 510 | 0.108 +/− 0.015 | 1300 |
| GCC | 0.209 +/− 0.025 | 600 +/− 67 | 0.084 +/− 0.008 | 1500 |
| GTG | 0.175 +/− 0.011 | 610 | 0.659 +/− 0.169 | 11 +/− 5.6 |
| GAG | 0.151 +/− 0.020 | 670 | 0.120 +/− 0.063 | 690 |
| TCT | 0.146 +/− 0.023 | 690 | 0.083 +/− 0.009 | 1500 |
| ACG | 0.143 +/− 0.030 | 690 | 0.063 +/− 0.009 | 1700 |
| ATG | 0.136 +/− 0.037 | 720 | 0.069 +/− 0.007 | 1600 |
| CTC | 0.122 +/− 0.009 | 760 | 0.058 +/− 0.006 | 1800 |
| GGG | 0.118 +/− 0.021 | 770 | 0.122 +/− 0.024 | 1100 |
| CCC | 0.119 +/− 0.017 | 770 | 0.063 +/− 0.014 | 1700 |
| CAG | 0.105 +/− 0.008 | 820 | 0.056 +/− 0.010 | 1800 |

The mutant RGPD was selected from the phage display library using the DNA sequences GCG and GCT, while the mutant REDV was selected using GCG and GTG. The microarray binding results not only verify binding to these respective sequences, but also indicate that RGPD binds fairly well to CCG and to GCT (though RGPD was not recovered from a selection using CCG). These results demonstrate that microarray binding experiments can be used to distinguish the DNA binding site preferences of transcription factors, even those with highly overlapping DNA binding specificities. In addition, the binding site preferences of these two mutants may-differ even further, since it has been noted that the 5′ nucleotide of the triplet binding site of finger 1 is also contacted by finger 2 (M. Isalan, Y. Choo, A. Klug, PNAS USA 94, 5617 (1997). Microarray binding experiments using an expanded set of DNA binding sites corresponding to all possible 4-mer binding sites for finger 2 may further distinguish the binding site preferences of Zif268 variant.

These microarray binding experiments appear to indicate that wildtype Zif268 has a broader spectrum of DNA binding site specificity than do tightly binding Zif268 mutants isolated from in vitro selections. One hypothesis is that wildtype Zif268 has evolved to bind a number of different DNA sites, so as to regulate various genes using a wide range of interaction strengths, whereas in in vitro selection the only function that the mutant must retain is reasonably tight binding to the target sequence. Another hypothesis that can be derived from these microarray binding experiments is that rounds of selection using the wildtype DNA target site TGG would yield mutants that are more specific for TGG than are the wildtype Zif268 fingers.

Distinguishing Binding Site Preferences—Ex. III: NonSpecific Binding

Given the utility of the method, the binding site preferences of zinc fingers which previously had been poorly characterized were determined. The mutants LRHN and KASN (Single-letter abbreviations for the amino acid residues are as follows: A, Ala, alanine; C, Cys, cysteine; D, Asp, aspartic acid; E, Glu, glutamic acid; P, Phe, Phenylalanine; G, Gly, glycine; H, His, histidine; I, Dle, isoleucine; K, Lys, lysine; L, Leu, leucine; M, Met, methionine; N, Asn, asparagine; P, Pro, proline; Q, Gln, glutamine; R, Arg, arginine; S, Ser, serine; T, Thr, threonine; V, Val, valine; W, Trp, tryptophan, Y, Tyr, Tryrosine) had been isolated repeatedly after independent sets of in vitro selections using many different triplet binding sites for the second finger (ACT, AAA, TTT, CCT, CTT, TTC, AGT, CGA, CAT, AGA, AGC, and AAT). Although the basis for the outcome of these selections was not clear, it was apparent that most of the above triplets had A or C present at the 5′ nucleotide position. These triplet binding sites also had failed to select any sequence-specific zinc fingers from the phage library. Although LRHN was isolated in all of these selections, microarray binding experiments revealed that this variant is fairly specific for the DNA sequence containing the triplet binding site TAT (Table 1C).

TABLE 1C
LRHN DNA-binding Results

| sequence of finger 2 | | |
|---|---|---|
| DNA binding site | relative signal intensity | K_{d}^{app} (nM) |
| TAT | 1 | 6.3 +/− 1.6 |
| GAT | 0.500 +/− 0.085 | 72 |
| AGT | 0.449 +/− 0.095 | 93 |
| AAT | 0.607 +/− 0.195 | 110 +/− 39 |
| TAG | 0.300 +/− 0.080 | 110 +/− 31 |
| CAT | 0.429 +/− 0.144 | 120 +/− 26 |
| TGT | 0.384 +/− 0.052 | 130 |
| ACT | 0.342 +/− 0.073 | 160 |
| CGT | 0.330 +/− 0.071 | 170 |
| TAC | 0.329 +/− 0.130 | 170 |
| CCT | 0.298 +/− 0.062 | 200 |
| ATT | 0.274 +/− 0.085 | 240 +/− 24 |
| GGT | 0.238 +/− 0.032 | 260 |

Moreover, the LRHN-TAT complex is almost as tight as the wildtype Zif268-DNA complex in these experiments. Meanwhile, microarray binding experiments showed KASN to be fairly nonspecific, with binding to a number of DNA sequences consistent with the middle triplet consensus (A/C/T)NT (Table 1D).

TABLE 1D
KASN DNA-binding Results

| sequence of finger 2 | | |
|---|---|---|
| DNA binding site | relative signal intensity | K_{d}^{app} (nM) |
| AAT | 1 | 250 +/− 28 |
| CCT | 0.974 +/− 0.272 | 250 |
| ACT | 0.949 +/− 0.415 | 260 |
| ATT | 0.942 +/− 0.225 | 270 |
| TAT | 0.911 +/− 0.270 | 270 +/− 28 |
| AGT | 0.862 +/− 0.115 | 320 |
| TGT | 0.856 +/− 0.232 | 320 |
| CGT | 0.848 +/− 0.188 | 330 |
| CGC | 0.774 +/− 0.260 | 390 |
| TAG | 0.773 +/− 0.178 | 390 +/− 40 |
| GAA | 0.697 +/− 0.172 | 1000 |
| CGG | 0.655 +/− 0.244 | 1460 +/− 170 |
| GAC | 0.652 +/− 0.077 | 1300 |
| GGC | 0.636 +/− 0.144 | 1400 |
| GGT | 0.616 +/− 0.279 | 1600 |
| GGG | 0.612 +/− 0.103 | 1700 |

These results indicate that microarray binding experiments are highly sensitive in determining the DNA binding site preferences of DNA binding proteins, as the K_{d}^{app} of the tightest KASN complex is over 80 times weaker than the interaction of wildtype Zif268 with its optimal binding site. A clone chosen at random from the starting library was applied to a microarray. No binding was detectable for any of the triplet sequences on the microarray. This suggests that a majority of the variants in the starting library are not capable of binding DNA at micromolar or lower affinities, even nonspecifically, even though they have retained wildtype finger 1 and finger 3 amino acid sequences.

These experiments highlight the importance of using a binding site weight matrix in describing the preferential binding sites of transcription factors, over the use of either the optimal or consensus binding sites. The use of an optimal or consensus binding site would not distinguish the binding preferences of the variants RGPD and REDV. A more complicated scheme might assign a penalty for every position that differs from the consensus binding site. However, such a system would predict wildtype Zif268 to have a weaker binding affinity for the triplet binding site GAG than for TCG, while microarray binding experiments have determined the opposite ordering to be correct. Therefore, it is a binding site weight matrix, constructed using K_{d}^{app}'s, that describes the binding site preferences of transcription factors more accurately. Once these types of microarray binding experiments are performed using an expanded set of binding sites, a complete reference table of the affinities of a given transcription factor for all possible binding sites will be available that should virtually eliminate the need for a consensus or weight matrix representation of the binding site preferences.

Metrics of Binding Specificity

These experiments can also distinguish highly related proteins based upon their affinities for sub-optimal DNA sites. Both ranking preferences and distinguishing by suboptimal affinity were considered in calculating three types of DNA binding specificity scores as metrics of how specific a protein is for its most tightly binding DNA site, as well as of the general spectrum of affinities that the protein has for variant DNA sites (Table 2). These analyses show that the Zif268 wildtype protein and variants perform very differently in site-specific DNA recognition.

Objective metrics of DNA binding specificity are computed from the data. In particular, three parameters of DNA binding specificity (S1, S2, S3) are of utility in describing the affinity of a protein for its most tightly bound DNA site relative to the general the general spectrum of affinities that the protein has for variant DNA sites (Table 2). S1, the single substitution score, is defined as:

$$S 1 = \frac{{\tilde{K}}_{d , ss}^{app}}{{K}_{d , bt}^{app}} \qquad ( 1 )$$

wherein

$${K}_{d , bt}^{app}$$

is the apparent K_{d} of binding to the best triplet (bt) and

$${\tilde{K}}_{d , ss}^{app}$$

is the average of the nine single substitutions (ss) of the best triplet. Thus, if the best triplet is GCT,

$${\tilde{K}}_{d , ss}^{app}$$

is the average of the apparent K_{d} for binding to ACT, CCT, TCT, GGT, GAT, GTT, GCA, GCG, GCC, the single substitution being underlined.) S2, the apparent K_{d} range, is defined as:

$$S 2 = \frac{{K}_{d , lt}^{app}}{{K}_{d , bt}^{app}} \qquad ( 2 )$$

wherein

$${K}_{d , lt}^{app}$$

is the apparent K_{d} of binding to the lowest affinity triplet (lt). The K_{d}^{app} range is the ratio of affinity for the lowest affinity binding site to the affinity for the highest affinity binding site. As such it serves as a measure of the selectivity of a protein. S3, the overall preference, is defined as:

$$S 3 = \frac{{\tilde{K}}_{d , N0Tbt}^{app}}{{K}_{d , bt}^{app}} \qquad ( 3 )$$

wherein

$${\tilde{K}}_{d , N0Tbt}^{app}$$

is the average apparent K_{d} of binding for all sites except the best (bt) or highest affinity site.

TABLE 2
DNA binding specificity scores.

| | S1: single | | |
|---|---|---|---|
| variant | substitution score | S2: K_{d}^{app} range | S3: overall preference |
| REDV | 511 | 1110 | 920 |
| wildtype | 31.1 | 78.3 | 66.2 |
| RGPD | 37.7 | 74.7 | 65.2 |
| LRHN | 39.6 | 89.1 | 74.1 |
| KASN | 2.75 | 5.09 | 3.23 |

DNA binding specificity scores. The S1 or single substitution score for a protein variant is defined as the mean K_{d}^{app} of the 9 possible single base pair substitutions of the triplet with the highest binding affinity, divided by the K_{d}^{app} of the triplet with the highest binding affinity. The S2 or K_{d}^{app} range is defined as the K_{d}^{app} of the triplet with the lowest DNA binding affinity, divided by the K_{d}^{app} of the triplet with the highest DNA binding affinity. The fluorescence intensities of spots at or below background were set to be the standard deviation of the spot with the lowest quantifiable fluorescence intensity on the respective microarrays. The K_{d}^{app} range serves as a measure of the specificity of a variant for the triplet with the highest binding affinity versus the triplet it binds most weakly. The final score, S3, is a measure of the overall preference of a variant for the triplet with the highest binding affinity versus any other triplet binding site.

These analyses show that the Zif268 perform very differently in site-specific DNA recognition relative to the middle finger variants Cable 2). For example, the REDV variant is much more selective than wild-type by all three parameters, whereas the KASN is hardly selective by these standards. These binding specificity scores provide objective criteria that can predict whether a protein binds at only a few specific sequences in a genome or if it binds more uniformly.

Whole Genome Analysis—Mouse

The wildtype Zif268 binding site matrix based on the microarray binding experiments was used to search the available mouse genome sequence for potential new genomic binding sites for Zif268 that had not yet been described (P. P. Roth, J. D. Hughes, P. W. Estep, G. M. Church, Nature Biotechnol. 16, 939 (1998)). The publicly available mouse genome sequence was downloaded from the NCBI website at http://ray.nlm.nih.gov/genome/seq/MmHome.html. This sequence was then searched using the program ScanACE. Although 24 examples of the sequence GCGTGGGCG were found, the sequence GCGTAGGCG was not found at all. Of the 24 genomic examples of the GCGTGGGCG binding site, 5 are located within 2 kb upstream (4 of these 5 are located within 620 bp upstream) of an mRNA start codon, 7 are located between exons, 1 is found 128 bp downstream of a gene, and 3 are found within coding regions; the remaining 8 sites are not annotated.

This could indicate that although GCGTAGGCG has an K_{d}^{app} almost as tight as that of GCGTGGGCG, these genomic sites have mutated over time to GCGTGGGCG so as to bind Zif268 with a stronger binding affinity. Another explanation could be that another transcription factor binds competitively to a site that overlaps the wildtype Zif268 site, and a TAG triplet would hinder binding of that transcription factor. Alternatively, a genomic example of this binding site simply has not been found yet, as only 1% (32 Mb) of the mouse genome has been sequenced so far. Assuming the GC content of the mouse genome to be 40%, only 3.7 GCGTAGGCG sites and only 2.5 GCGTGGGCG sites are expected to occur by chance in 3.2 Mb of sequence. Nevertheless, this type of approach for constructing binding site matrices with which to search genomes for potential new genomic binding sites should prove useful for discovering new target sites for transcription factors in genomes for which more sequence is available.

Whole Genome Analysis—Drosophila

For example, a whole genome Drosophila melanogaster microarray consisting of roughly 27,000 spots covering a total of 120 Mb genes could be used to characterize the DNA binding specificities of over 670 D. metanogaster transcription factors, at least 135 of which are zinc finger proteins (M. Adams, et al., Science 287, 2185 (2000); FlyBase, at http://ray.nlm.nih.gov/genome/seq/MmHome.html). Of the roughly 13,600 genes in D. melanogaster, over 670 (4.9%) are transcription factors and at least 135 (1%) are zinc finger proteins. Extrapolating these gene ratios to estimate the number of transcription factors and zinc finger proteins in the human genome, there are thousands of transcription factors, approximately one thousand of which are zinc finger proteins, which could be characterized using this methodology (E. Pennisi, Science 288, 1146 (2000)). There are an estimated 29,000-153,000 genes in the human genome. Therefore, 4.9% of 29,000-153,000=1421-7497 transcription factors, and 1% of 29,000-153,000=290-1530 zinc fingers.

Library Bias Detection

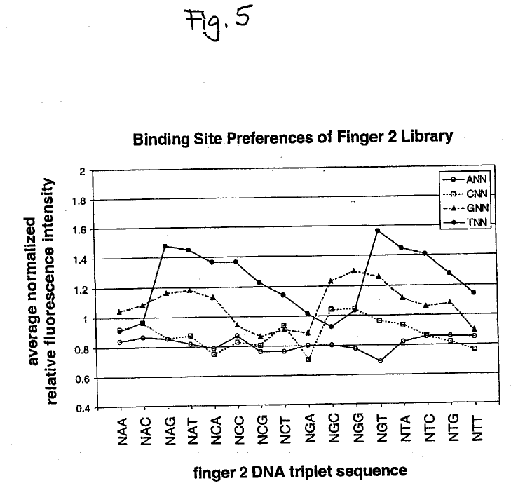

The zinc finger library's bias against binding DNA triplets with 5′ A or C was tested using microarrays in order to determine if the library bias could have been deduced prior to selection experiments. Therefore, the entire library of zinc finger clones was applied to a microarray, and analyzed binding of the entire population to the 64 different triplets. In general, triplets with a 5′ T were bound significantly better than their counterparts with a 5′ G, which in turn were bound significantly better than their counterparts with a 5′ A or C (T>G>A/C) (FIG. 5). The reason for this is that the 5′ nucleotide of the middle triplet is potentially specified by a combination of contacts from fingers 2 and 3 (see base pair 5 in FIG. 1A), but the library has only the residue from finger 2 randomized. At the time the library was made, the Zif268-DNA cocrystal structure indicated that the three zinc fingers recognized individual 3 bp subsites. However, since then it has been determined that the zinc fingers recognize overlapping 4 bp subsites. This means that the 5′ base of the central triplet is partially specified by an amino acid from finger 3, which was not randomized in the library. This amino acid imposes a preference for T or G in the 5′ position of the middle triplet (Asp 2 of finger 3 accepts a hydrogen bond from the NH₂ group of A or C on the Crick strand). The library is therefore biased towards binding sequences that have the specificity of wildtype Zif268 for the 5′ nucleotide of the middle triplet, i.e., T, or to a lesser degree G. The use of microarrays to determine the DNA sequence preferences of entire libraries of DNA binding domains will be extremely useful in designing libraries of better quality, and (assuming multiple libraries are available) for determining which library is best suited to selection using a particular DNA sequence.

To see if the evolution of sequence-specific phage from library selections could be followed, pools of library members eluted from different panning rounds of the GCG selection were bound to microarrays (FIG. 3A). Specifically bound sequences could be detected starting with round 2. The sensitivity of this method is limited by the standard deviation of the fluorescence signal due to nonspecific binding. Assuming that the apparent nonspecific binding constants are identical for all sequences, assigning a cutoff of 3 standard deviations above the signal due to nonspecific binding assures that specific binding is called with 99.9% confidence. A significant change in the DNA binding preference of the population occurred during the third round of selection, from T(A/T)G in round 2, to G(C/T)G in round 3. There was very little change in the DNA binding specificity of the population during the fourth round of selection. These microarray binding data are a useful aid to carrying out successful phage selections and can be used to guide the improvement of selection conditions or to determine the endpoint of a selection.

As a further demonstration of the utility of this approach for tracking the evolution of binding site specificity during phage selections prior to sequencing of the selected phage, rounds 1 through 3 of the TCC selection were applied to microarrays, since this selection appeared to fail to produce zinc fingers specific for TCC (FIG. 3B). Sequencing analysis of clones obtained from this selection revealed that the experiment had not yielded zinc fingers specific for TCC, but had instead produced zinc finger clones that were also selected by the triplet GAC. Without the use of sequence information for the selected zinc fingers, microarray binding experiments of phage pools from rounds 1 through 3 indicate that the selection was driven by zinc finger phage that bind to the sequence present at the GAC spot on the microarrays. These phage were selected because the sequence GCGGACGCA is the complement of TGCGTCCGC, which closely resembles the sequence of the DNA used in the TCC selection. GCGGACGCA is present on the complementary strand of the DNA sequence at the TCC spot on the microarrays, offset by 1 bp from the intended register of the triplet binding sites (FIG. 3C). The microarray results also indicate that GCGGACGCG (the top strand of the GAC spot) is bound more tightly by the phage pool eluted from round 3 of the TCC selection than are either GCGTCCGCG (the top strand of the TCC spot) or GCGGACGCA (the bottom strand of the TCC spot), which are present on the Watson and Crick strands, respectively, at the TCC spot on the microarrays. Formally, the microarray binding experiments indicate only which sequences are bound. However, in the case of the well-studied Zif268-like zinc fingers it is possible to deduce the binding sites within these DNA sequences. The AT-rich sequences flanking the 9 bp binding sites for the Zif phage serve as an attempt to confine zinc finger binding to within the GC-rich portion.

A Representation of an Interaction Site Profile

An interaction site profile can be a list containing as many objects as capture probes. For example, an array in which a 3 basepair interaction site is varied to all combination of basepairs at that site has 64 capture probes. The probes include nucleic acids with the 3 basepair sequence TIT, ATT, . . . etc, as illustrated briefly in column 1 of Table 3. Each object in the interaction site profile has associated value, e.g., x₁, x₂, etc, as illustrated briefly in column 2 of Table 3.

TABLE 3

| Object | Associated Value |
|---|---|
| TTT | x₁ |
| ATT | x₂ |
| GTT | x₃ |
| CTT | x₄ |
| TAT | x₅ |
| AAT | x₆ |
| . | . |
| . | . |
| . | . |
| CCC | x₆₄ |

In an alternative embodiment, an interaction site profile can contain references to only those capture probes for which an interaction is observed. For example, a compound that only interacts with GTT and CIT has an interaction site profile as illustrated in Table 4. The length of length of such lists is expected to vary from compound to compound. In comparing such lists, objects are compared first to identify. The list can also be sorted, e.g., by rank order using the associated value. Thus the list can have the object representing a probe with the highest affinity for a compound first.

TABLE 4

| Object | Associated Value |
|---|---|
| GTT | x₁ |
| CTT | x₂ |

Other embodiments are within the following claims.

What is claimed:

1. A method of providing an interaction site profile for a compound comprising providing an array of a plurality of capture probes, wherein each of the probes in the plurality is positionally distinguishable from other probes of the plurality, and wherein each positionally distinguishable probe includes a unique region;
contacting the compound with the array; and
identifying probes to which the compound interacts thereby providing an interaction site profile.

2. The method of claim 1 wherein the interaction site profile is a list of objects, each object representing a unique capture probe, and having an associated value.

3. The method of claim 2 wherein the list comprises a plurality of objects.

4. The method of claim 3 wherein the list comprises a plurality of objects, each unique capture probe being represented by an object.

5. The method of claim 2 wherein a plurality of the associated values in the list are different.

6. The method of claim 2 wherein the associated value is a function of the amount of interaction between the compound and the probe.

7. The method of claim 6 wherein the associated value is a function of the amount of binding between the compound and the probe.

8. The method of claim 1 wherein the interaction between the compound and the nucleic acid is a binding interaction.

9. The method of claim 3 wherein the interaction site profile is stored in computer memory or on computer readable media.

10. The method of claim 1 wherein the compound is a polypeptide.

11. The method of claim 10 wherein the polypeptide is a transcription factor.

12. The method of claim 11 wherein the transcription factor binds a double stranded DNA sequence with an affinity of 10 mM or less.

13. The method of claim 11 wherein the transcription factor is selected from the group consisting of homeodomains, helix-tum-helix motif proteins, beta-sheets, leucine zippers, steroid receptors, zinc fingers and histones.

14. The method of claim 10 wherein the polypeptide is a zinc finger polypeptide.

15. The method of claim 10 wherein the polypeptide is covalently attached to a bacteriophage.

16. The method of claim 10 wherein the polypeptide is linked with an unrelated sequence.

17. The method of claim 10 wherein the polypeptide is covalently attached to green fluorescent polypeptide.

18. The method of claim 10 wherein the polypeptide comprises a detectable label.

19. The method of claim 10 wherein the polypeptide is contacted with an antibody.

20. The method of claim 15 wherein the bacteriophage is contacted with an antibody.

21. The method of claim 10 wherein the polypeptide is a variant of a natural counterpart, the variant having at least one amino acid difference from the natural counterpart.

22. The method of claim 21 wherein the differing amino acid is located within 50 Ångstroms of the bound nucleic acid in a structural model.

23. The method of claim 1 wherein the capture probes are nucleic acids selected from the group consisting of, double-stranded DNA, single-stranded DNA, RNA, PNA, or hybrids thereof.

24. The method of claim 23 wherein the nucleic acids are double stranded DNA (dsDNA).

25. The method of claim 23 wherein the nucleic acids comprise at least 15 basepairs.

26. The method of claim 25 wherein the nucleic acids comprise at least 30 basepairs.

27. The method of claim 23 wherein the unique region of the nucleic acids comprises a plurality of basepairs.

28. The method of claim 1 wherein the plurality of capture probes comprises at least 48 species.

29. The method of claim 28 wherein the plurality of capture probes comprises at least 64 species.

30. The method of claim 29 wherein the plurality of capture probes comprises at least 128 species.

31. The method of claim 29 wherein the plurality of probes comprises all possible combinations of natural basepair substitutions at greater than two basepairs of the interaction site.

32. The method of claim 30 wherein the plurality of probes comprises all possible combinations of natural basepair substitutions at greater than three basepairs of the interaction site.

33. The method of claim 1 wherein the array is a solid silica support and the plurality of nucleic acid probes are stably attached to the support.

34. The method of claim 33 wherein the array is a solid silica support to which a nucleic acid probes are stably attached by an amino linkage.

35. The method of claim 1 wherein the nucleic acid probes comprise genomic DNA.

36. The method of claim 35 wherein the nucleic acid probes comprises non-coding genomic DNA.

37. A method of evaluating a plurality of compounds comprising:
(1) providing a plurality compounds,
(2) providing an array of a plurality of capture probes, wherein each of the probes in the plurality is positionally distinguishable from other probes of the plurality, and wherein each positionally distinguishable probe includes a unique region;
(3) contacting each compound with an array (e.g., the same array, or a different array);
(4) identifying probes to which each compound interacts thereby providing an interaction site profile for each compound; and
(5) comparing the interaction site profiles to thereby evaluate the plurality compounds.

38. The method of claim 37 wherein the interaction site profile is a list of objects, each object representing a unique capture probe, and having an associated value, which is a function of the amount of compound bound to the probe.

39. The method of claim 38 wherein two interaction site profiles are compared by providing a difference profile that consists of a list of objects which are common to both interaction site profiles, and associating with each object in the difference profile a value which is a function of the values associated with the corresponding objects in the two interaction site profiles being compared.

40. The method of claim 39 wherein two interaction site profiles are compared by providing a value which is a function of at least one of the values in the difference profile.

41. The method of claim 37 wherein two interaction site profiles are compared by comparing one or a plurality of the associated values in the two interaction site profiles.

42. The method of claim 37 wherein two interaction site profiles are compared by comparison to a reference profile.

43. The method of claim 37 wherein the interaction site profile is stored in computer memory or on computer readable media.

44. A method of evaluating a first polypeptide comprising:
a) providing one or a plurality of reference polypeptides;
b) providing a first polypeptide;
c) obtaining the interaction site profiles for the first polypeptide and at least one reference polypeptide, at least one interaction site profile being provided by
i) providing an array of a plurality of nucleic acid probes, wherein each of the probes in the plurality is positionally distinguishable from other probes of the plurality, and wherein each positionally distinguishable probe includes a unique region;
ii) contacting a polypeptide (e.g., the first polypeptide, one or more reference polypeptides) with the array of probes; and
iii) identifying probes to which the polypeptide interacts and thereby providing an interaction site profile; and
d) comparing the interaction site profile of each reference polypeptide with the interaction site profile of the first polypeptide to thereby evaluate the first polypeptide.

45. The method of claim 44 further comprising:
identifying a selected reference polypeptide from the plurality such that the interaction site profiles of the selected reference polypeptides meets a predetermined level of similarity with the interaction site profile of the first polypeptide; and
assigning to the first polypeptide the function of selected reference polypeptide.

46. The method of claim 44 wherein the interaction site profile is a list of objects, each object representing a unique nucleic acid probe, and having an associated value, which is a function of the concentration of compound bound to the probe.

47. The method of claim 46 wherein two interaction site profiles are compared by calculating a difference profile that consists of the same list of objects as the interaction site profiles and that has associated with each object a numerical value which is a function of the two interaction site profiles.

48. The method of claim 47 wherein two interaction site profiles are compared by calculating a numerical score which is a function of at least one, of the numerical values in the difference profile.

49. The method of claim 46 wherein two interaction site profiles are compared by ordering the objects in each interaction site profile by their associated value, and comparing the relative ordered position of an object that occurs in both interaction site profiles.

50. The method of claim 46 wherein the interaction site profile is stored in computer memory or on computer readable media.

51. The method of claim 44 wherein the reference polypeptides are transcription factors.

52. The method of claim 44 wherein the reference polypeptides are mammalian.

53. The method of claim 51 wherein the reference polypeptides are zinc fingers.

54. The method of claim 44 wherein the reference polypeptides comprise variants of a naturally occurring polypeptide.

55. The method of claim 44 wherein the nucleic acid probes are double stranded DNA.

56. The method of claim 55 wherein the plurality of nucleic acids comprises at least 48 species.

57. A method of selecting a polypeptide comprising:
(1) providing a plurality of polypeptides;
(2) contacting the plurality with a substrate comprising target nucleic acid;
(3) isolating a selected population from the plurality;
(4) providing an array of a plurality of nucleic acid probes, wherein each of the probes in the plurality is positionally distinguishable from other probes of the plurality, and wherein each positionally distinguishable probe includes a unique region which corresponds to a binding site for the polypeptide, and wherein at least one probe of the plurality comprises the target sequence;
(5) contacting the selected population with the array;
(6) identifying probes to which the selected population interacts thereby identifying the interaction site profile of the selected population;
(7) evaluating the interaction site profile for a predetermined condition; and
(8) isolating a polypeptide from the selected population, thereby selecting a polypeptide.

58. The method of claim 57 optionally repeating steps (2) to (7) until the predetermined condition is met.

59. The method of claim 57 wherein the predetermined condition is an affinity for the target nucleic acid.

60. The method of claim 57 wherein the target sequence is degenerate.

61. The method of claim 60 wherein the substrate comprises more than one species of nucleic acid.

62. The method of claim 57 wherein the plurality of polypeptides comprises members of a library constructed from expressed genes (cDNA).

63. The method of claim 57 wherein the plurality of polypeptides comprises variants of a progenitor polypeptide.

64. The method of claim 63 wherein the variants differ by at least one amino acid whose side chain is within 10 Ångstroms of the nucleic acid binding interface.

65. The method of claim 63 wherein the variants are members of a library generated by a method selected from the group consisting of: cassette mutagenesis, PCR mutagenesis, and altered genetic codes.

66. The method of claim 63 wherein the polypeptide variants are derived from transcription factors.

67. The method of claim 63 wherein the polypeptide variants are derived from a mammalian polypeptide.

68. The method of claim 63 wherein the polypeptide variants are derived from zinc fingers.

69. The method of claim 57 wherein the nucleic acids are double stranded DNA.

70. The method of claim 57 wherein the plurality of nucleic acids comprises at least 48 species.

71. The method of claim 57 wherein the plurality of nucleic acids comprises at least 64 species.

72. The method of claim 57 wherein the plurality of nucleic acids comprises at least 128 species.

73. The method of claim 71 wherein the nucleic acid probes comprise the complete set of mutations of at least 3 base pair positions.

74. The method of claim 57 wherein the substrate comprises a nucleic acid coupled to a bead.

75. The method of claim 57 further comprising comparing the interaction site profile with an interaction site profile of a previous iteration; and terminating the selection procedure if the differences between the interaction site profiles are not substantial.

76. The method of claim 57 wherein the criteria in step 7 further comprises evaluating the apparent binding affinity of the selected population for the target sequence.

77. The method of claim 76 wherein the apparent binding affinity is a function of the interaction site profile.

78. A polypeptide produced by the method of claim 57.

79. A method of selecting a polypeptide with a predetermined criterion, (e.g., an interaction with DNA, a DNA binding site specificity) comprising:
(1) providing a plurality of polypeptides,
(2) identifying the interaction site profile of each polypeptide of the plurality by the method of claim 1; and
(3) selecting the polypeptide whose interaction site profile meets the predetermined criterion thereby selecting a polypeptide with a predetermined criterion.

80. The method of claim 79 wherein the plurality of polypeptides consists of variants of a common reference polypeptide.

81. The method of claim 80 wherein the polypeptide variants differ from the common reference polypeptide by no more than 10 amino acid alterations.

82. The method of claim 80 wherein the amino acid alterations are no more than 10 Ångstroms away from the nucleic acid interaction site in a structural model of the interaction between the common reference polypeptide and the nucleic acid interaction site.

83. The method of claim 79 wherein the plurality of polypeptides consists of polypeptides encoded by expressed genes.

84. A polypeptide produced by the method of claim 79.

85. A method of designing a polypeptide to bind a desired DNA binding site comprising:
providing a reference protein with at least two domains that contact DNA;
providing a plurality of variants in each domain, the variants being different from a reference domain by at least one amino acid in the interface which contacts DNA;
determining the interaction site profiles for each of the plurality of variants by the method of claim 1;
selecting, for each domain, variants whose interaction site profile manifests specificity for a fragment of the desired DNA binding site;
linking selected variants of each domain to provide at least one candidate polypeptide;
determining the interaction site profiles for each candidate polypeptide by the method of claim 1; and
selecting candidate polypeptides whose interaction site profile indicates specificity for a desired DNA binding site, thereby selecting a polypeptide with a desired DNA binding site specificity.

86. A method of evaluating a plurality of polypeptides comprising:
(1) providing a plurality of polypeptide variants,
(2) providing an array of a plurality of nucleic acid probes, wherein-each of the probes in the plurality is positionally distinguishable from other probes of the plurality, and wherein each positionally distinguishable probe includes a unique region which corresponds to a binding site for the polypeptide;
(5) contacting the plurality of polypeptides with the array of probes,
(6) identifying probes to which the plurality of polypeptides interacts thereby identifying an interaction site profile; and
(7) assessing the interaction site profile for interactions for desired nucleic acid sequences to thereby evaluate the plurality of polypeptides.

87. A method of screening a nucleic acid sequence for the presence of candidate sites for a compound comprising:
providing a interaction site profile for a compound, by the method of claim 3;
providing a nucleic acid sequence;
selecting interaction sites from the interaction site profile, the selected interaction sites having an associated value that meets a preselected requirement; and
indicating the presence or absence of the selected interaction sites in the nucleic acid sequence to thereby screen a nucleic acid sequence for candidate sites for interaction with a compound.

88. The method of claim 87 wherein the nucleic acid sequence is genomic nucleic acid sequence or a fragment thereof.

89. The method of claim 87 wherein the interaction site profile and the nucleic acid sequence are stored in computer memory and/or on computer readable medium.

90. A database of interaction sites comprising:
a plurality of records, at least one record referencing a nucleic acid sequence, the sequence being a candidate site identified by the method of claim 87.

91. A database of interaction sites comprising:
a plurality of records, at least one record referencing a hit to an external database, the hit specifying a candidate site identified by the method of claim 87.

92. A computer program product comprising a computer-useable medium having a computer-readable program code embodied thereon, the code for effecting:
accepting an interaction site profile;
accessing a database of nucleic acid sequence; and
providing candidate sites in the accessed database by the method of claim 87 for the accepted interaction site profile.

93. A computer readable media comprising one or a plurality of interaction site profiles produced by the method of claim 3.

94. A database, stored on computer readable media or in computer memory, comprising a plurality of records, the records been a reference to a compound and a reference to the interaction site profile of the compound, the profile being provided by the method of claim 3.

95. A computer system comprising the database of claim 94, and a user interface capable of receiving a reference to a compound and of providing an interaction site profile.

96. A method of predicting the sites bound by a compound in a genome or fragments thereof comprising:
evaluating the level of active compound molecules in a cell;
obtaining the interaction site profile of the compound by the method of claim 1;
determining the number of occurrences in the nucleic acid sequences of the genome or fragments thereof for each of the plurality of sites in the interaction profile to thereby predict the interactions sites bound by a compound in a cell.

97. The method of claim 88 further comprising: determining the probability for each of the plurality of sites that a compound is interacting with the site.

98. The method of identifying a regulatory protein for a plurality of coregulated genes comprising:
providing a regulatory nucleic acid sequence for each member of the plurality;
providing a set of interaction site profiles for a set of reference proteins;
identifying for each reference protein candidate interaction sites within the regulatory nucleic acid sequence of each member of the plurality of coregulated genes by the method of claim 87;
selecting the reference proteins that have candidate interaction sites for a number of coregulated genes, the number being greater than a threshold value to thereby identify a regulatory protein for a plurality of coregulated genes.

99. The method of claim 1 wherein the array comprises capture probes, the probes having a unique region, and each species of probe being present at a plurality of positionally distinguishable locations such that the concentration of probe at each of the plurality of locations differs.

100. A method of providing interaction site profiles for a compound comprising:
providing samples of a compound at a plurality of compound concentrations; and
providing interaction site profiles for each sample by the method of claim 1 to thereby provide interaction site profiles for a compound.

101. The method of claim 1 wherein the step of identifying probes to which the compound interactions is repeated after a time interval to provide a plurality of interaction site profiles for a compound.
